(19) United States
(12) Patent Application Publication
(10) Pub. No.: US 2013/0224822 A1
(43) Pub. Date: Aug. 29, 2013

(54) MODIFICATION OF MICROALGAE FOR MAGNETIC PROPERTIES

(71) Applicants: Richard Sayre, Webster Groves, MO (US); Brad Postier, Saint Charles, MO (US)

(72) Inventors: Richard Sayre, Webster Groves, MO (US); Brad Postier, Saint Charles, MO (US)

(21) Appl. No.: 13/808,746

(22) PCT Filed: Jul. 7, 2011
(86) PCT No.: PCT/US11/43192

§ 371 Date: May 15, 2013

Related U.S. Application Data
(60) Provisional application No. 61/362,160, filed on Jul. 7, 2010.

(51) Int. Cl. C12N 13/00

(52) U.S. Cl. CPC C12N 13/00
USPC 435/173.9; 435/257.2; 435/320.1

(57) ABSTRACT

Genetically engineered algae strains for biofuels and bioproduct production have improved iron utilization including iron uptake and storage, and exhibit improved growth characteristics, magnetic separation, and magnetic hysteresis induced cell lysis. Pond production algal strain embodiments with high iron scavenging capabilities limit iron availability to contaminating microorganisms and invading species. Accumulation of high iron and other paramagnetic elements content in the form of ferritin enhances the cells magnetic susceptibility improving efficiency of magnetic separation and magnetic hysteresis induced cell lysis. Several genes of the embodiments are capable of improving iron acquisition including the ferritin gene fer1, iron transport gene fea1, and iron reductase gene fre1. Other genes which improve growth in the high iron conditions permitting higher accumulation of iron include radical scavenging enzymes such as superoxide dismutase, peroxidase, catalase, glutathione peroxidase and the Ferritin like DPR/DPS genes which also bind iron and protect DNA from reactive oxygen species.

### CROSS-REFERENCE TO RELATED APPLICATIONS

This application is based on and claims priority to U.S. provisional patent application Ser. No. 61/362,160 filed Jul. 7, 2010, the teachings of which are included herein by reference.

### TECHNICAL FIELD

The disclosed embodiments of the present invention are in the field of algal biomass and biofuel production.

### BACKGROUND

Biofuels will play an increasing role in the United States energy market as energy prices increase, political concerns of establishing energy independence intensify, and apprehension about climate change grows. The price of petroleum has fluctuated dramatically, reaching record highs of more than US$140 per barrel in 2008. In part, those price increases reflected economic, political, and supply chain uncertainties. Political concerns about the availability of petroleum supplies have led to the realization that the United States' energy independence is of critical strategic importance, both economically and militarily. Additionally, the release of CO₂ from fossil fuel combustion may also substantially contribute to global warming and climate change and that could have environmental and societal importance. Many states and federal governments have responded by enacting renewable portfolio standards (RPS) mandating that electricity providers and other heavy energy users obtain a certain percentage of their power from renewable energy sources. As a result of these concerns, sustained high prices for fossil fuels, and the emerging RPS requirements, domestically produced biofuels have become an increasingly attractive alternative to foreign sourced fossil fuels.

Microalgae are some of the most productive and therefore desirable sources of biofuel feedstocks. The Department of Energy (DOE) has determined that biofuel yield per acre from microalgae culture exceeds that of many organisms and land crops. Between the late 1970s and 1990s, the DOE's National Renewable Energy Laboratory (NREL, formerly SERI) evaluated the economic feasibility of producing biofuels from a variety of aquatic and terrestrial photosynthetic organisms (Sheehan et al., 1998). Biofuel production from microalgae was determined to have the greatest yield per acre potential of any of the organisms screened. Microalgal biofuel production was estimated to be 8 to 24 fold greater than the best terrestrial biofuel production systems. Current estimates of the potential productivity for algal biofuel production range from 3,000 to 10,000 gallons/acre. According to the DOE, microalgae yield “30 times more energy per acre than land crops such as soybeans.” Although promising, there is still a need for systems and methods that create even greater efficiencies in biofuel production from microalgae.

One of the challenges faced by biofuel production from microalgae is that, traditionally, the organisms must be dewatered to reduce the overall processing volume and improve the efficiency of the extraction process. Currently available dewatering processes, however, have many drawbacks. For instance, centrifugation and filtration are costly and labor intensive, making them somewhat impractical for production of commodity products. Settling, in either holding tanks or plate type settling devices, is an inexpensive though time-consuming process (U.S. Pat. No. 3,431,200; Kothardaraman & Evans 1972). This method is ill-suited for dewatering algae because of the small size and low specific gravity of algal cells. The settling rate for algae is therefore “too slow to permit the use of a settling as a routine procedure for harvesting algae cells” (Kothandaraman & Evers 1972). The length of the settling process for algae poses several difficulties, especially in a production plant where the cells being collected will either be used in a downstream process or for extraction of oil. First, the long processing time decreases plant throughput. Second, the long processing time increases the likelihood of contamination that can compromise downstream processing (U.S. Pat. No. 3,431,200). The long term storage of the cells associated with settling can lead to decay or reabsorption of the product of interest (e.g., oil, pigments, secondary metabolites, and other co-products) for cell maintenance that can compromise oil extraction efficiency and yields. In addition, because of the large amount of water involved, centrifugation is cost prohibitive.

Dissolved air flotation (DAF) is another separation method somewhat related to the settling method previously described (U.S. Pat. No. 3,780,471; U.S. Pat. No. 3,431,200; Kothandaram & Evans 1972). Under this method, the cells are treated with flocculants and/or coagulating agents. The cells then aggregate and are exposed to a fine curtain of air bubbles that lift aggregated cells to the top of the holding tank where they are skimmed off and harvested. The utility of this method is limited because coagulated and flocculated cells can compromise downstream processing. Removing the coagulants or flocculants involves another processing step which increases total processing time and costs. Although faster than simple settling, this method still has the same drawbacks associated with any slow process for dewatering to downstream processing and extraction.

A pitfall of all of these methods is that their dewatered products contain organisms other than algae. The presence of these organisms can compromise the efficiency of downstream processing. For instance, some of these organisms may out-compete algae, thereby decreasing the density of the targeted algae.

Dewatering can be achieved magnetically if the material to be separated is sufficiently magnetized. Commercially available magnetic separators operate by allowing the magnetized target particle to attach directly to a rotating magnetic disc(s). As the disc is rotated, one section is continuously scrubbed by a cell scraper that removes the adhered biomass and transfers it to a conveyor belt or pipe where it is distributed for downstream processing. Contaminating species, nonmagnetic algae, and debris flow preferentially to waste or further treatment such that it can be sent back to the pond for further growth. An alternative use would be to use the magnetic properties of the production strain to positively select and return it to the production process while removing contaminating microorganisms from the culture on a continuous or periodic basis as a maintenance or emergency response protocol.

In order for magnetic separation to be effective, however, the materials to be separated must be sufficiently magnetized. Existing methods of magnetization are limited to altering the exterior of the material to be separated. One such method is magnetic seeding, which involves the addition of magnetite to the outside of the material with or without the use of a sorption agent (U.S. Pat. No. 7,473,356; Niki et al.). Magnetic separation of this type of seeded material has drawbacks. This method causes all materials to coagulate, including undesirable materials such as fungi, protozoa, bacteria, archaea, and other species of algae. As mentioned previously, the presence of these contaminating materials may out-compete the desired algae, decreasing the yield and efficiency of the lipid production process.

Unless otherwise defined, all technical and scientific terms used herein have the same meaning as commonly understood by one of ordinary skill in the art to which this invention pertains. Although methods and materials similar or equivalent to those described herein can be used in the practice or testing of the exemplary embodiments, suitable methods and materials are described below. In case of conflict, the present specification, including definitions, will control. In addition, the materials, methods, and examples are illustrative only and not intended to be limiting.

Microalgae have been variously defined through the ages and it is prudent to describe the microalgae to which this invention could apply. For the purposes of this patent, microalgae include the traditional groups of algae described in Van Den Hoek et al. (1995). The subject application is applicable to, for example and without limitation, cultures of macroalgal seaweeds and all other algae which form filaments or other structures that could be helped during harvest by this invention. It is noted that this definition includes the cyanobacteria, traditionally referred to as bluegreen algae, which are prokaryotic in nature even though the majority of algal strains are eukaryotic in nature.

The terminology as set forth herein is for description of the embodiments only and should not be construed as limiting the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such. The singular “alga” is likewise intended to be inclusive of the plural “algae.”

The phrase “genetically modified algae,” as used herein, refers to algae whose genetic material has been altered using genetic engineering techniques so that it is no longer a “wild type” organism. An example of genetically modified algae is transgenic algae that possess one or more genes that have been transferred to the algae from a different species. Another example is an alga wherein endogenous genes have been rearranged such that they are in a different and advantageous arrangement or amplified so that specific sequences are increased. In this example, no foreign DNA remains in the modified cell.

The phrase improved productivity or enhanced productivity of algae can refer to increases in oil content per cell, increased cell number per unit volume, increased cell size, increased areal productivity, various combinations of these, or other bio-product produced per unit area per time interval.

### SUMMARY OF THE EMBODIMENTS

These and other unmet needs of the prior art are met by exemplary compositions and methods as described in more detail below.

Exemplary embodiments of the compositions, systems, and methods disclosed herein improve the process of producing biofuels and bioproducts from microalgae. This is achieved by enhancing the natural magnetic susceptibility of algal cells through genetic engineering and taking advantage of this magnetic property to improve the processing of algal biomass. These improvements include the separation of algal biomass from liquid media, and induction of cell lysis through magnetic hysteresis. The systems and methods provided include obtaining an algal strain(s) transformed by using an expression vector to improve iron utilization. Methods for utilization of said strains include improved growth conditions in open production ponds, improved dewatering through magnetic separation, and efficient product extraction through improved cell lysis via magnetic hysteresis. In one embodiment, for example, excess iron is stored in the form of ferritin engineered to be expressed in greater quantities than the wild-type strain which confers the enhanced magnetic susceptibility of the host cell.

In one aspect, genetically engineered algae strains have resultant improved iron utilization for biofuels production for improved growth characteristics, magnetic separation, and magnetic hysteresis induced cell lysis. In open pond production, algal strains of the embodiments with high iron scavenging capabilities limit iron availability to contaminating microorganisms and invading species. Accumulation of high iron and other paramagnetic elements content in the form of ferritin enhances the cells magnetic susceptibility. Enhanced magnetic susceptibility improves the efficiency of magnetic separation and magnetic hysteresis induced cell lysis. Several genes are capable in the embodiments for improving iron acquisition including the ferritin gene Fer1, the iron transport gene Fea1, and the iron reductase gene Fre1. Other genes which improve growth in the high iron conditions thus permitting higher accumulation of iron in accordance with the embodiments include gene encoding, radical scavenging enzymes such as superoxide dismutase, peroxidase, catalase, glutathione peroxidase and the ferritin-like DPR/DPS genes which also bind iron and protect DNA from reactive oxygen species. Gill and Tetuja Plant Physiol. Biochem. 2010 December; 48(12):909-30 and Chiacone and Ceci, Biochim. Biophys. Acta 2010 August; 1800(8):798-805.

In a further aspect, embodiments of the present invention involve genetically modifying algal strains so that a gene encoding the ferritin protein is overexpressed and accumulates in the cell's cytoplasm, chloroplast(s), or mitochondria.

Another embodiment of the present invention involves genetically modifying algal strains so that an iron transporter and iron reductase are overexpressed simultaneously, allowing more iron to be transported into the cell.

In yet another embodiment of the present invention, contamination control and separation efficiency are enhanced through precise iron dosage and feeding strategies during the cultivation of algae genetically modified to over-accumulate ferritin complexes.

In another embodiment of the present invention, the enhancement of magnetic susceptibility makes recombinant algae more susceptible to magnetic hysteresis. When exposed to a magnetic field of reversing polarity at high frequency (100-400 kHz), the magnetic poles on each paramagnetic element in the accumulated magnetic particles (Ferritin for example) switch orientation along with the field. Internal resistance to high frequency dipole switching can induce heat generation and may be sufficient to induce cell lysis, thus liberating the oil contained within the cell. Such an embodiment would provide significant cost advantages to the extraction of oil from these modified algae.

In another embodiment of the present invention, paramagnetic or ferromagnetic particles/elements besides iron may be assimilated into biomass to improve the magnetic susceptibility of the cell as a whole. These may include but are not limited to: manganese, lithium, magnesium, aluminum, tin, calcium, titanium, cobalt, nickel, tungsten, and neodymium. These elements may be incorporated into functional biomass, locked into storage mechanisms, or transported into the vacuole.

In another embodiment of the present invention, strains may be improved to tolerate high concentrations of ferromagnetic or paramagnetic particles present within the cell during photosynthetic growth. This may be achieved by overexpression of genes that provide resistance to the specific stresses induced by the accumulation of one or more of the various elements (or similar) described above. For example the overaccumulation of iron can have deleterious effects on algal growth by interacting with some components of the photosynthetic apparatus. Reactive oxygen species can be produced by the Photosystem I complex under high light conditions. When these reactive oxygen species interact with iron they become even more reactive as free radical compounds (example: Fe²⁺+H₂O₂→Fe³⁺+OH+OH⁻) and more toxic to a broader variety of sites within the cell. Genes encoding proteins that protect against reactive oxygen species would thus enable growth under conditions such that iron can overaccumulate safely within the cell. Some examples of radical scavenging enzymes are catalase, peroxidase, superoxide dismutase, glutathione peroxidase and the ferritin like protein DPS or DPR which bind and store iron, and bind to DNA. The DPR/DPS complexes serve to protect DNA from damage by free radicals.

In yet another embodiment of the present invention, strategies to reduce the fluidic drag on cells during magnetic separation can enhance magnetic separation. There are two forces competing against magnetic separation. The first is gravity. Gravity pulls on the cell in a constant direction causing settling at the bottom of any reactor or separation chamber. The second is fluidic resistance. As cells move in a column of water toward a magnetic pole or surface, the rate at which cells move is a function of the strength of the magnet, the paramagnetic moment of the cells and the drag forces of the fluid the cells are suspended in. Reducing these drag forces can improve the efficiency of magnetic separation, reduce the time of magnetic separation, and increase flow rates. This improved efficiency can be achieved by maintaining a high ratio of magnetic moment/cell volume. That is, a small cell will be easier to separate if it has the same iron content as a larger cell as the fluid drag on the larger cell is greater and works against magnetic separation. It may also be possible to engineer genetic elements that reduce the drag coefficient of the algal surface through modification of glycosylation patterns or altering the composition of the cell wall

### BRIEF DESCRIPTION OF THE DRAWINGS

A better understanding of the exemplary embodiments of the invention will be had when reference is made to the accompanying drawings, and wherein:

FIG. 1a shows an example construct of a gene encoding ferritin protein (fer1) linked to the RuBisCo (rbcL) promoter and ATP synthase β subunit (atpB) terminator for improving the magnetic properties of algae.

FIG. 1b shows an example construct of a gene for iron transport (fea1) expressed from an actin promoter and terminator for improving the magnetic properties of algae.

FIG. 2 is a schematic illustration showing iron homeostasis under normal conditions as controlled by ferritin, iron transporters, and iron reductases.

FIG. 3 is a schematic illustration showing high iron homeostasis achieved through routes including over-expression of the ferritin gene and resulting complexes, over-expression of the iron transporter and required iron reductase; and increasing the availability of iron in the medium surrounding these genetically modified algae through controlled and precise iron dosing in accordance with example embodiments.

FIG. 4 is a schematic illustration showing magnetic separation based on an increased magnetic susceptibility due to increased iron storage in ferritin complexes in accordance with an example embodiment.

FIG. 4a is a chart illustrating mean velocities of algae cells in a uniform magnetic field such as the field of FIG. 4.

FIG. 5 is a schematic illustration showing magnetic separator system in accordance with an example embodiment which operates by allowing the target magnetically susceptible particle to attach directly to a rotating magnetic disc(s).

FIG. 6 is a schematic illustration showing magnetically induced cell lysis of algae genetically modified to over-accumulate ferritin complexes in accordance with the example embodiments.

FIG. 7 is a graphical illustration showing enhancement of contamination control and separation efficiency through precise iron dosage and feeding strategies in accordance with the example embodiments.

FIG. 8 is a graphical illustration showing isolates of iron replete cells grown in iron deficient medium in accordance with example embodiments.

FIG. 9a is a chart illustrating relative growth of algae in accordance with example embodiments in iron replete and in iron depleted conditions compared to wild-type algae.

FIG. 9b is a bar chart illustrating bacterial contamination in algal growth in accordance with example embodiments in iron replete and in iron depleted conditions and as against wild-type algae.

FIG. 10 is a growth chart illustrating effects of strains upon transfer to low iron media.

FIGS. 11a and 11b are growth charts showing an impact of overexpression of multiple genes involved in iron assimilation on growth in media containing elevated iron.

### DETAILED DESCRIPTION

With reference now to the drawings wherein the showings are for purposes of illustrating the example embodiments only and not for purposes of limiting same, using the variety of methods of the exemplary embodiments of the invention, the example embodiments described herein are directed to improved algal cells and at improving the extraction of desirable compounds from algal cells. The example embodiments are particularly applicable to magnetic separation of microalgae modified to safely assimilate iron in the form of paramagnetic particles from liquid media.

### Genetic Modification of Microalgae to Enhance Magnetic Susceptibility

Ferritin, a protein existing in nearly all living organisms that store iron, is expressed in microalgae. Ferritin is an iron storage protein complex consisting of >4000 iron atoms arranged such that the complex becomes paramagnetic. However, the amount of ferritin naturally occurring in microalgae is conditionally variable but typically insufficient to permit magnetic separation of algae from liquid media under normal growing conditions. Cells containing sufficient amounts of ferritin bound iron (either internally or externally labeled) demonstrate a response when exposed to a magnetic field. In a suspension, this response is to move toward one pole or another of the magnetic field. As discussed above, magnetic separation of algae from their liquid medium is desirable because it is less costly and time- and labor-intensive than conventional separation methods. To employ this method of separation, however, the amount of iron in algal cells must be increased such that the cells are sufficiently magnetically susceptible.

A specific range of intracellular iron levels is required for normal growth and reproduction. If iron levels fall outside this range, either above or below, growth is slowed. To buffer the bioavailability of intracellular iron, ferritin complexes capture and store excess iron, and subsequently release iron as necessary for optimal growth. Iron homeostasis under normal conditions is controlled by ferritin, iron transporters, and iron reductases.

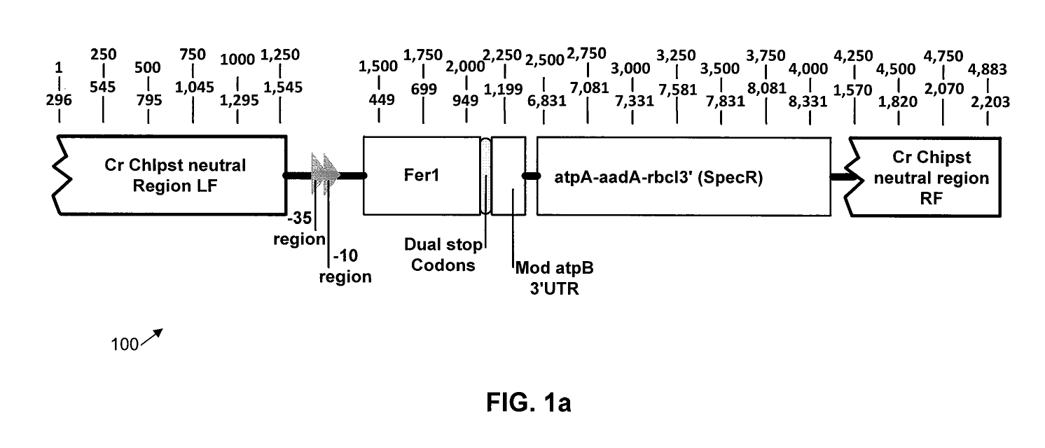

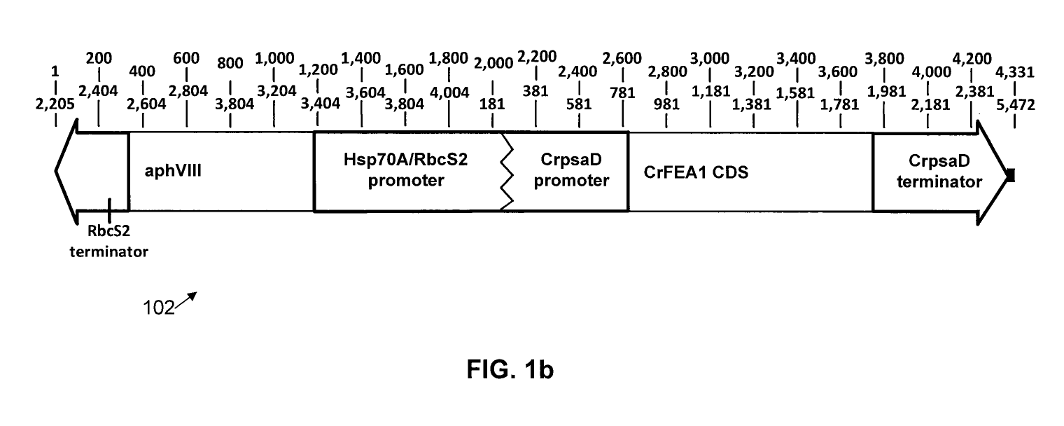

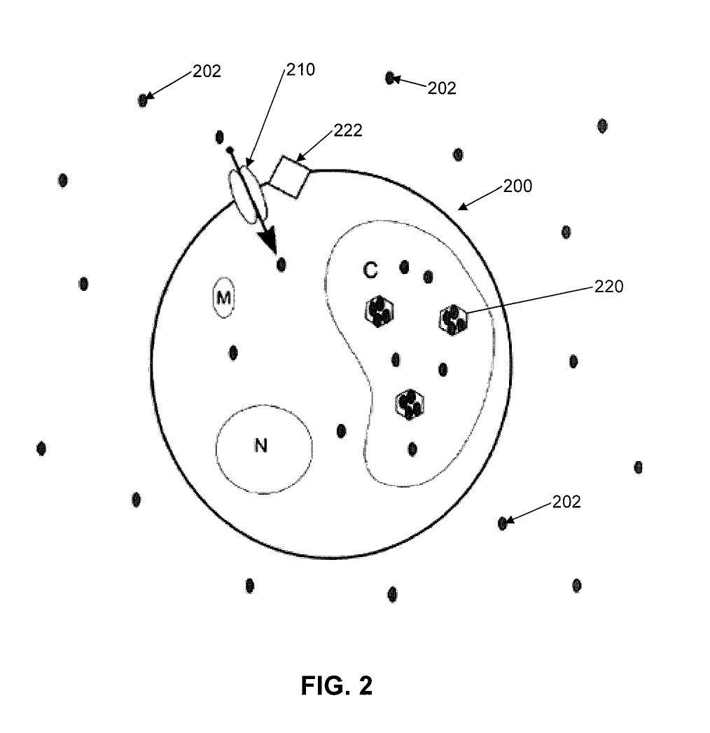

With reference now in particular to FIGS. 1a and 1b, ferritin is nuclear encoded in algae and transported to the chloroplast for incorporation into ferritin complexes. Photosynthetic iron requirements in the chloroplast are high, giving the storage of iron in the chloroplast an evolutionary advantage over nuclear, cytoplasmic or mitochondrial localization as it permits a faster response to homeostatic iron levels. The nuclear fer1 gene encodes ferritin in Chlamydomonas reinhardtii (Long & Merchant, Photochemistry and Photobiology, 84: 1395-1403 (2008)). Increased storage of iron, over wild type cells, can be therefore achieved by expressing the fer1 gene from Chlamydomonas reinhardtii in the chloroplast genome under the control of the RuBisCo large subunit promoter and atpB 3′ UTR as seen in FIG. 1a.

In this case, the described fer1 gene encoding ferritin is located on a plasmid adjacent to a selective marker. Together these two elements are flanked by regions of sequence homology to an insertion site in the chloroplast genome of interest enabling double crossover homologous recombination. This recombination will successfully insert the selectable marker and the targeted ferritin gene into the chloroplast genome. Other potential promoters and terminators may be used such as those for photosynthesis core proteins, 16S rRNA, and chlorophyll biogenesis. Further alternative promoters may be inducible such that the magnetic properties only present themselves when necessary to reduce the burden of the increased iron requirements. These inducible promoters may be activated by light, specific carbohydrates, salt shock, heat stress, or other signal molecules that could be applied to a production environment. The iron homeostasis is then balanced by increased activity of the iron transporter and coupled iron reductase. Ferritin production in cells can also be increased by overexpressing the genes for iron transporters and iron reductases from the constitutively active highly expressed actin promoter. Known Chlamydomonas reinhardtii iron transporters include FTR1, FEA1, FEA2, IRT1, and IRT2 (Fei et al., J. of Biomedicine and Biotechnology, 2010, 1-9 (2010); Allen et al., Eukaryotic Cell, Vol. 6, No. 10: p. 1841-1852 (2007)). Iron reductases include fre1 which may encode a ferrireductase that aides the transport of iron by reduction of Fe³⁺to Fe²⁺(Long et al., Genetics Soc. Of Am., 179: p. 137-147 (2008)). These genes can use their own native terminator or a terminator native to the target host. As shown in FIG. 1b, fea1 is expressed from the constitutively active highly expressed actin promoter. As with the FER1 example embodiment of FIG. 1a, alternative promoters, such as those that are activated by the presence of certain compounds or stressors, may make this system inducible.

Chloroplast genomes of Chlamydomonas reinhardtii have been transformed by biolistic bombardment with gold particles containing linearized plasmids carrying at least two kinds of genes and two flanking elements. The two flanking elements are regions of DNA taken from the host strain that allow double crossover homologous recombination. This is a naturally occurring process in algal chloroplasts that permits the introduction or recombination of DNA elements into a targeted site. For instance these two flanking regions may come from a contiguous region between genes. In this case, the insertion of the genetic material between the two flanking regions on the plasmid will be inserted into a “neutral region” on the chromosome. In another scenario it may be necessary to interrupt or “knock out” a gene by using two flanking regions comprised of DNA sequence identical to sequence from a target gene or from flanking regions around a target gene to be displaced. Based on scientific literature, roughly 600 nt of identical sequence in both flanking regions is sufficient for efficient transformation.

The first of the two genes used for inducing magnetic susceptibility is a selectable marker. This marker gene can be comprised of an antibiotic resistance gene such as the aadA gene conferring resistance to spectinomycin or it could be the reintroduction of a previously displaced gene such as psbA or other required native gene. This selectable marker allows the easy identification of colonies after transformation through growth on selective medium (containing spectinomycin or under photoautotrophic growth respectively for the two example marker genes). Regardless of the gene used as a marker it will be necessary to ensure moderate expression. This is achieved through the use of chloroplast appropriate 5′ and 3′ UTRs. Native genes expressed from a chloroplast genome use a typical prokaryotic promoter element with or without a −35 region. Typical 5′UTRs contain a −10 region (TATAATAT) around 10 nucleotides upstream of the +1 nt of the transcript (transcript initiation site) followed closely by a ribosome binding site (GGCC). A 5′UTR roughly 100 nt in length is sufficient for moderate expression. Typically 5′UTRs from native genes such as atpA, atpB, psbA, psbB, rpoA, or other specific gene are used as promoter elements. 3′ UTR regions contain a stop codon (or pair of stop codons) followed by an AT rich region of ˜20 nt and a hairpin forming region of DNA of roughly 25 nt in length. This stops translation and transcription and stabilizes the transcript for full activity. Stopping transcription is required when there are concerns of read through of downstream genes.

The second gene used for this construct is the gene conferring the magnetic property. In this example that target gene is ferritin. The native ferritin from the C. reinhardtii nuclear genome is sufficient but will require trimming of the N terminus sequence to its mature form for expression from the chloroplast and codon optimization to that preferred by the target chloroplast. (Joanne C. Long, 2008). In this case the ferritin gene is expressed using the rbcL 5′ UTR and atpB 3′ UTR for expression at high levels in the chloroplast as seen in FIG. 1. Over-expression of the FER1 protein produces ferritin complexes in abundance allowing significantly enhanced iron storage capability. Use of native ferritin allows normal iron homeostasis, but with increased storage capacity. Iron stored in ferritin complexes are paramagnetic and thus confer the magnetic properties necessary for separation, and magnetic hysteresis.

The DNA sequence used to generate the Fer1 over-expression strain 100 illustrated in FIG. 1a and described above is, in the example embodiment, as set out as follows (in fasta format).

>Fer1 overexpression vector sequence for transformation of Chlamydomonas reinhardtii chloroplastic genome:

CCCTTTTGGCAGGACGTCCCTTTCCCCTTACGGGATATTTATATACTCC
CATGTTTTGCCCTTTACGGGACGCCAGTGGACGTCCCCTTCCCCTTCCG
GGCAAGTAAACTTAGGGATTTTAATGCAATAAATAAATTTGTCCCCTTA
CGGGAATATAAATATTAGTGGCAGTTGCCTGCCAACTGCCGAGGCAAAT
AAATTTTAGTGGACAAATTTATTTATTGTTGCCTGCCAACTGCCGATAT
TTATATACTCCGAAGGAGGCAGTTGCCGGCCAACTGCCTGCCCCGCAGT
ATTAACATAAGCAGTGTCGGTACCATTGCCACTGGCGTCCCGTTAGGTG
TTCGTAGCAGGCAGTGTCCTGCCACTGCCTCCTGTGGAGTATATAAATA
TCCCTAAGTGTACTTGCCCGTAAAGGGAAAGGGAAGGGTATTTTATTTA
CTTTTTAGGTTTAGTAATAAATGAATCACAGGTAATTTCACTTTACATG
CCATAAACTCTCTAATATACGTAAAGCTTCGCTCTGCGCAAGACTAAAG
TGCCCAAGCTCAAAGAACCTTTTCAAGAGTAAATAAATTCCCTTAAGGG
CTAATGAGCAAATTTAATGGTTATAGTTTTGTCAAGAAACGTTAGCACT
GTTAGTACCAGGTTTAAAACATTTTTAATTTTTAATAATGCCAACAAAC
AATGTCGATGTAAGAAATGGATTTATTTTTTTAATAAATTTAATTTTAT
GCGTGAATCAATTCGAAGCGAATATGAAGTGAGCGGTTCAAAAAGAACA
ACTAACTATGGCTTTAGATATGTGAAATATCTATATAACTAAATTGCAC
ACCCTTTTGTAAATAGAACCTAAAAAGGAAAAAAATTGAATTTAATAAA
TGACCCCTCAGCCAAAAGGTATGATTGGCAGTGCCTTTACTCGATAAAC
CCTTTGGAGATTTAATTGCCCTTAAAAGAAAAGACTAAAAATGTTTGCT
ACCCAGGCAGTAGCGGGATTTGAAATACTCCGTACAGCAGACGTCCCCT
TACGGGACGTCAGTGGATATTTATATACTCCGAAGGAGGCAGTTGCCTG
CCAACTGCCGATATTTATATACTGCGATAAACTTTAGTTGCCCGAAGGG
GTTTACATACTAGGCAGTGGCGGTACCACTGCCACTGGCGTCCTCCTTC
GGAGTATGTAAACTACTTGCCCGAAGGGGAAGGAGGAAGCAGGCAGTGG
CGGTACCACTGCCCCTTACGGGATCCAATTGACGGGATCCGAATTCCGT
GAGCTCACGCGATCGCGAGGTACCCATATGACTAGTCATCTAGATAAAT
TTGCTAGTTTACATTATTTTTTATTTCTAAATATATAATATATTTAAAT
GTATTTAAAATTTTTCAACAATTTTTAAATTATATTTCCGGACAGATTA
TTTTAGGATCGTCAAAAGAAGTTACATTTATTTATATAAATGGACAAAA
TCACTGGCATTGTTGTTCAGCCTGCTGTTCAGTTCTCTGAAGTTCAGTC
TGAACTTGCTACTGTTGATAAAACTAACCAGAACATCCAGTCACTTGCA
CGTGTTGACTTCCACCCAGCTTGTGAAGCAGCTATCAACGAACAGGTAA
ACATCGAATACAACGTTTCTTACCTATACCACGCTCTTTGGGCATACTT
CGACCGTGACAACGTAGCTCTACCTGGCCTTGCTGCTTTCTTCAAAGCT
GGCTCTGAAGAAGAACGTGAACACGCAGAACTTCTTATGGAATACCAGA
ACCGTCGTGGCGGCCGTGTTGTTCTAGGCGCAATCTCTATGCCTGACCT
AGACCTTTCTGCTTCTGAAAAAGGCGACGCTCTTTACGCAATGGAACTT
GCTCTTAGCCTAGAAAAACTTAACTTCCAGAAACTTCGTCAGCTTCACT
CTGTTGCAGACGAACACGGCGATGCTTCTATGGCTGACTTCGTAGAAGG
CGAATTATTAAACGAACAGGTTGAAGCTGTTAAAAAAGTTTCAGAATAC
GTATCTCAGCTTCGTCGTGTTGGCCAGGGCCTAGGCGTTTACCAGTTTG
ACAAACAGCTTGCAGCTGAAGTAGCAGCTGGTGCTGCAGCTTAATAACT
CGAGCCCGGGATGTGTAATTAAAATAAATATTTGGACACCATTAAGTTG
TTTTCTTCTTAAAGAGCCAATTTATTTTAATTACACATACATTATATAT
GTCATATTAGCTATTTAATCTATTTACTTTAAAATAAAGGACGCCGTTA
AACGTGTACGCGTGCATCGGCTGGTACCATGCATGCGGCCGCAGTAGAC
TTTATTAGAGGCAGTGTTTATATACCTAAACGTCAAAAGTCATTTTTAT
AACTGGTCTCAAAATACCTATAAACCCATTGTTCTTCTCTTTTAGCTCT
AAGAACAATCAATTTATAAATATATTTATTATTATGCTATAATATAAAT
ACTATATAAATACATTTACCTTTTTATAAATACATTTACCTTTTTTTTA
ATTTGCATGATTTTAATGCTTATGCTATCTTTTTTATTTAGTCCATAAA
ACCTTTAAAGGACCTTTTCTTATGGGATATTTATATTTTCCTAACAAAG
CAATCGGCGTCATAAACTTTAGTTGCTTACGACGCCTGTGGACGTCCCC
CCCTTCCCCTTACGGGCAAGTAAACTTAGGGATTTTAATGCAATAAATA
AATTTGTCCTCTTCGGGCAAATGAATTTTAGTATTTAAATATGACAAGG
GTGAACCATTACTTTTGTTAACAAGTGATCTTACCACTCACTATTTTTG
TTGAATTTTAAACTTATTTAAAATTCTCGAGAAAGATTTTAAAAATAAA
CTTTTTTAATCTTTTATTTATTTTTTCTTTTATGGCAATGCGTACTCCA
GAAGAACTTAGTAATCTTATTAAAGATTTAATTGAACAATACACTCCAG
AAGTGAAAATGTCCATGGCTCGTGAAGCGGTGATCGCCGAAGTATCGAC
TCAACTATCAGAGGTAGTTGGCGTCATCGAGCGCCATCTCGAACCGACG
TTGCTGGCCGTACATTTGTACGGCTCCGCAGTGGATGGCGGCCTGAAGC
CACACAGTGATATTGATTTGCTGGTTACGGTGACCGTAAGGCTTGATGA
AACAACGCGGCGAGCTTTGATCAACGACCTTTTGGAAACTTCGGCTTCC
CCTGGAGAGAGCGAGATTCTCCGCGCTGTAGAAGTCACCATTGTTGTGC
ACGACGACATCATTCCGTGGCGTTATCCAGCTAAGCGCGAACTGCAATT
TGGAGAATGGCAGCGCAATGACATTCTTGCAGGTATCTTCGAGCCAGCC
ACGATCGACATTGATCTGGCTATCTTGCTGACAAAAGCAAGAGAACATA
GCGTTGCCTTGGTAGGTCCAGCGGCGGAGGAACTCTTTGATCCGGTTCC
TGAACAGGATCTATTTGAGGCGCTAAATGAAACCTTAACGCTATGGAAC
TCGCCGCCCGACTGGGCTGGCGATGAGCGAAATGTAGTGCTTACGTTGT
CCCGCATTTGGTACAGCGCAGTAACCGGCAAAATCGCGCCGAAGGATGT
CGCTGCCGACTGGGCAATGGAGCGCCTGCCGGCCCAGTATCAGCCCGTC
ATACTTGAAGCTAGACAGGCTTATCTTGGACAAGAAGAAGATCGCTTGG
CCTCGCGCGCAGATCAGTTGGAAGAATTTGTCCACTACGTGAAAGGCGA
GATCACTAAGGTAGTTGGCAAATAACAGCTTGTACTCAAGCTCGTAACG
AAGGTCGTGACCTTGCTCGTGAAGGTGGCGACGTAATTCGTTCAGCTTG
TAAATGGTCTCCAGAACTTGCTGCTGCATGTGAAGTTTGGAAAGAAATT
AAATTCGAATTTGATACTATTGACAAACTTTAATTTTTATTTTTCATGA
TGTTTATGTGAATAGCATAAACATCGTTTTTATTTTTTATGGTGTTTAG
GTTAAATACCTAAACATCATTTTACATTTTTAAAATTAAGTTCTAAAGT
TATCTTTTGTTTAAATTTGCCTGTGCTTTATAAATTACGATGTGCCAGA
AAAATAAAATCTTAGCTTTTTATTATAGAATTTATCTTTATGTATTATA
TTTTATAAGTAATAAAAGAAATAGTAACATACTAAAGCGGATGTAACTC
AATCGGTAGAGTGCGATCGCTAGCACTAGTCTCGAGGTTTAAACATGTA
CAAGCTTGGATCAATTGATTGGCTGCTGTGCAGCCCCAAAGTAGAAGGG
TACGCCAGTGGACGTCAGTGGCAGGCAACTGCCTCCTTCGGAGTATATA
AATATTGGCAGTTGGCAGGCAACTGCCACTGACGTCCACTTAAATTTAT
TTGCCCGAAGGGGACGTCCACTGGCGTACCGTAAGGGGAAGGGGACACC
CACTGGCGTCCCGAAAGGATTAGCCAATGGGCGAGGCCACTGCTTCAGC
TACTAAAGTTTTAAAGTGGTCTGTTGGACCATCAAATTCATATGGCATG
TAATACTCCTAACGCTAATGCCTGGCTTTAGGGTCAGTTTACTGACCCA
AAAACCTGTTAATAGCTTGTTAAACCCTTCTTTCCCTTTTGGAGTATAT
CACTATCACAAAGGGGCGTCAGATGGAGTTAGTAAACTTTTTAAAGTGA
ACAAGAATCACAGATGTAATAAGCTTATTAAGTAAAGCTTAGAAAAATT
GTTTTAATAACTAACAAAACTTTAAAAGTTCAAAAATTTTAATGTTATG
TATAATATTTTAATTAAAAAAACCATAACTATGTAAACCTTCTCCAATT
AAATTTACACCTAAATAACAAAACCAAACAACTAAAAAACCAACAGCAG
CAATAATTGCCGGTTTTTCACCTTCCCAACCA.

With reference to FIG. 1b, the Fea1 over-expression construct for Chlamydomonas reinhardtii using the psaD promoter and terminator with aphVII as the selective marker in accordance with a further embodiment (in fasta sequence) is as set out below. The vector for over-expression of the fea1 gene in Chlamydomonas reinhardtii is based on pSL18 derived from the work of Dèpege et al. (Role of Chloroplast Protein Kinase Stt7 in LHCII Phosphorylation and State Transition in Chlamydomonas in Science 7 Mar. 2003 pages 1572-1575). This vector contains an aphVII antibiotic resistance gene driven by the Hsp/RbcS2 promoter and RbcS2 terminator for selection on paromomycin. The fea1 gene was PCR amplified from cDNA of Chlamydomonas reinhardtii and cloned in a TOPO BluntII vector and subsequently sub-cloned in between the psaD promoter and terminator sequences of pSL18 as shown in FIG. 1B. The plasmid was linearized and introduced into Chlamydomonas reinhardtii strain CC2137 through biolistic transformation. Transformants were selected on Modified High Salt Agar plates with paromomycin as the selective agent. Isolated colonies were picked and screened for the presence of the introduced fea1 gene (the introduced fea1 gene can be differentiated from the wild type by the lack of intronic sequences). Positive transformants were then assayed for growth under low iron conditions. The sequence for the two DNA elements contained within the transformation vector required for conferring fea1 expression and supplying a positive selection marker in C. reinhardtii is as follows below.

>Fea1 nuclear expression vector sequence for the two components required for transformation of Chlamydomonas reinhardtii 102 is shown in FIG. 1b.

>Fea1 over-expression construct for Chlamydomonas reinhardtii using the psaD promoter and terminator with aphVII as the selective marker:

CTGGGTACCCGCTTCAAATACGCCCAGCCCGCCCATGGAGAAAGAGGCC
AAAATCAACGGAGGATCGTTACAACCAACAAAATTGCAAAACTCCTCCG
CTTTTTACGTGTTGAAAAAGACTGATCAGCACGAAACGGGGAGCTAAGC
TACCGCTTCAGCACTTGAGAGCAGTATCTTCCATCCACCGCCGTTCGTC
AGGGGGCAAGGCTCAGATCAACGAGCGCCTCCATTTACACGGAGCGGGG
ATCCCAACGTCCACACTGTGCTGTCACCCACGCGACGCAACCCTACCCA
GCCACCAACACCATCAGGTCCCTCAGAAGAACTCGTCCAACAGCCGGTA
AAACGCCAGCTTTTCCTCCGATACCGCCCCATCCCACCCGCGCCCGTAC
TCCCGCAGGAACGCCGCGGAACACTCCGGCCCGAACCACGGGTCCTCCT
CGTGGGCCAGCTCGCGCAGCACCAGCGCGAGATCGGAGTGCCGGTCCGC
ACGGCCGACCCGCCCCACGTCGATCAGCCCGGTCACCTCGCAGGTACGA
GGGTCGAGCAGCACGTTGTCCGGGCACAGGTGACCGTGGCAAACCGCCA
GATCCTCGTCCGCAGGCCGAGTCCGCTCCAGCTCGGCGAGAAGCCGCTC
CCCCGACCACCCCTTCCGCTCCTCGTCCAGATCCTCCAAGTCGACGCTC
CCTTCAGCGACAGCACGGGCCGCCTGCGGCACCGTCACCGCGAGACTGC
GATCGAACGGACACCGCTCCCAGTCCAGCGCGTGCAGCGAACGAGCGAG
CCCCGCGAGCGCCACCGCCACGTCCAGCCGCTGCTCCCGCGGCCACCGC
GCACTGGCCGGACGCCCCGGAACCGCTTCGGTGACCAACCAGGCGACCC
TCTCGTCCCCACCACCCTCCACAACACGAGGTACGGGAATCCCCACCTC
CGCCAACCACACCAGCCGCTCAGCCTCACCCAACAAGCCCACCCCGGCC
CCCAGAGCTGCCACCTTGACAAACAACTCCCGCCCACCACCCCGAAGCC
GATAAACACCAGCCCCCGAGGCCCCATCCTCCACAACAACCCACTCACA
ACCGGGATACCGACCCCGCAGTGCACGCAACGCATCGTCCATGCTTCGA
AATTCTTCAGCACCGGGGAGGGCGGAGTGGCCATCCTGCAAATGGAAAC
GGCGACGCAGGGTTAGATGCTGCTTGAGACAGCGACAGAGGAGCCAAAA
GCCTTCGTCGACACAATGCGGGCGTTGCAAGTCAAATCTGCAAGCACGC
TGCCTGATCCGCCGGGCTTGCTCGTCGACTCACCTGGCCATTTTAAGAT
GTTGAGTGACTTCTCTTGTAAAAAAGTAAAGAACATAGGCCCCCTGGCC
GGTTTATCAGGAGGGCACCGCTCCAGGGGCTGCATGCGAACTGCTTGCA
TTGGCGCCTAGCCTTTGTGGGCCAGGGGGCTTCCGGATAAGGGTTGCAA
GTGCTCAAATACCCCATCAAACATCATCCTGGTTTGGCTGCGCTCCTTC
TGGCGCGCCCGGCATGCAAGCTTGATGGGATCTTAAGCTAGCTGAGTGG
TTATGTATAGCGGCAGAATAGTCGCGTATGTATAAGTGCTCGTTTGTCG
CTGAAAGTGGAGGTCACCGTTCGGGGTCGCGGGCTTTTATACCGGATGG
GTGCCGCCAGCGGGCCGTATGGCGCCTTCTGGACGCCGCGCGCCCCATC
GCGGCCCTTCCAGAGCTTCCCGCGCCCTCATAGCCCGCCAAATCAGTCC
TGTAGCTTCATACAAACATACGCACCAATCATGTCAAGCCTCAGCGAGC
TCCCCGCTCGAGCACACACCTGCCCGTCTGCCTGACAGGAAGTGAACGC
ATGTCGAGGGAGGCCTCACCAATCGTCACACGAGCCCTCGTCAGAAACA
CGTCTCCGCCACGCTCTCCCTCTCACGGCCGACCCCGCAGCCCTTTTGC
CCTTTCCTAGGCCACCGACAGGACCCAGGCGCTCTCAGCATGCCTCAAC
AACCCGTACTCGTGCCAGCGGTGCCCTTGTGCTGGTGATCGCTTGGAAG
CGCATGCGAAGACGAAGGGGCGGAGCAGGCGGCCTGGCTGTTCGAAGGG
CTCGCCGCCAGTTCGGGTGCCTTTCTCCACGCGCGCCTNCCACACCTAC
CGATGCGTGAAGGCAGGCAAATGCTCATGTTTGCCCGAACTCGGAGTCC
TTAAAAAGCCGCTTCTTGTCGTCGTTCCGAGACATGTTAGCAGATCGCA
GTGCCACCTTTCCTGACGCGCTCGGCCCCATATTCGGACGCAATTGTCA
TTTGTAGCACAATTGGAGCAAATCTGGCGAGGCAGTAGGCTTTTAAGTT
GCAAGGCGAGAGAGCAAAGTGGGACGCGGCGTGATTATTGGTATTTACG
CGACGGCCCGGCGCGTTAGCGGCCCTTCCCCCAGGCCAGGGACGATTAT
GTATCAATATTGTTGCGTTCGGGCACTCGTGCGAGGGCTCCTGCGGGCT
GGGGAGGGGGATCTGGGAATTGGAGGTACGACCGAGATGGCTTGCTCGG
GGGGAGGTTTCCTCGCCGAGCAAGCCAGGGTTAGGTGTTGCGCTCTTGA
CTCGTTGTGCATTCTAGGACCCCACTGCTACTCACAACAAGCCCATATG
TCGGTCGGATTTCTGGTCCTCGCGCTGGGCGCGCTTGTCGTGGCGACGG
CGGACTACAAGGACGACGACGACAAGCAGCCCACGACGACTGGCACTCG
CTTCGAGGGTTTCTCTTACGCGGGCAATGTCATTGGCTATGTGAACATG
ACGATGGACTACTGCGACATCAAGGCCGCCATGGCTGCTGGCAACTTCA
CCGAGGCTCTGTCCATCTACTCTACTGGCAAGAACTCGTTCTCTGGCCT
GGCGCGCCGCACCTTCTTCCGCTTCGCCTCGTACATCACCGCCAACGGC
TCCGTGGAGCCGCTGCACGACTCCATCCTGGCCGGCAAGGACACGTCCT
CCCTGGACGCCGCCATCCGGGCTGCCCTGGCCGACGGCAAGGCCACCCT
GGCCGCCGGTCTGATCCAGGTGGGCACGCTCAAGTACCACCTGCACGAG
GTGGATGAGGCCTACAACAAGATCAAGACCTACCTGGCTGACGGCACCG
GCAACCTCACCAACCTGGTTTCTGACGCCTCGGGCGCACCCCACAACGT
GGATGAGGCCTGGGCGCTGTGGGCCGGCGGCGCCGCCAACAACTGCGGC
ACCCTGTCCGGCTGGGCCTCCTCCCTGGGCGCCGCCATGGGCACCACCT
TCCTCGGCAAGAGCTATGTCAACACCGCCATGATCAACACCGTCAATGA
GATGCTGGCCGCCGCCCGCCTGTCTACCCTGAACATCCAAGCCTACGAC
GCCGCGCGCACGAACGAGGTCCGACTGCTGACCCTGCTGGGCCTGCAGG
GCGTGTCCGTGGCCGCGTACACCGCTGACGCCGCCGCCGCCTGCAAGCG
CCCCGCCGCCGAGGTGGAGGATGCCAAGACCATGATCGCCGTGCACTGG
GCGTACCTGGAGCCTATGCTCAAGCTGCGCAACTTCAAGGCCTCCGCCG
TGACCGAGCTGCACCACCAGCTCACCGCCTCCAAGCTGAGCTACAAGAA
GGTGGCCGCCGCGGTGAAGGGCGTGCTGTCGGCTATGGGCCGCCGCTCC
AGCGAGCTGGGTGCCCCGCAGTCCGCCATCATTGCCGCCAACTGGAAGT
GCAGCTCCAAGACCCTGCGCAGCATTGCGTAATCTAGATGGCAGCAGCT
GGACCGCCTGTACCATGGAGAAGAGCTTTACTTGCCGGGATGGCCGATT
TCGCTGATTGATACGGGATCGGAGCTCGGAGGCTTTCGCGCTAGGGGCT
AGGCGAAGGGCAGTGGTGACCAGGGTCGGTGTGGGGTCGGCCCACGGTC
AATTAGCCACAGGAGGATCAGGGGGAGGTAGGCACGTCGACTTGGTTTG
CGACCCCGCAGTTTTGGCGGACGTGCTGTTGTAGATGTTAGCGTGTGCG
TGAGCCAGTGGCCAACGTGCCACACCCATTGAGAAGACCAACCAACTTA
CTGGCAATATCTGCCAATGCCATACTGCATGTAATGGCCAGGCCATGTG
AGAGTTTGCCGTGCCTGCGCGCGCCCCGGGGGCGCAGTTTAGCTGACCA
GCCGTGGGATGATGCACGCATTTGCAAGGACAGGGTAATCACAGCAGCA
ACATGGTGGGCTTAGGACAGCTGTGGGTCAGTGGACGGACGGCAGGGGA
GGGACGGCGCAGCTCGGGAGACAGGGGGAGACAGCGTGACTGTGCAATG
CGGCCGCCACCGCGGTGGA

In addition to the above, to express the DPS gene (sync_{—}2856 from the Synechococcus strain CC9311) in the chloroplast of the green alga Chlamydomonas reinhardtii the gene sequence is positioned between the rbcL promoter and atpB terminator such as that described for the fer1 gene. This is then situated within a plasmid construct adjacent to a selective marker gene with a promoter and terminator sequence. These two elements are then situated between two regions of sequence of >600 nt in length showing identical sequence to two closely linked sequences in the targeted chloroplast genome. These two regions of sequence homology permit site directed insertion of the DPS and selective marker gene (aada in this example)into the chloroplast genome of Chlamydomonas reinhardtii. An example of how this sequence could be assembled is included in fasta format:

>DPS (Synechococcus CC9311) expression vector for Chlamydomonas reinhardtii chloroplast

GCCCTTTTGGCAGGACGTCCCTTTCCCCTTACGGGATATTTATATACTC
CCATGTTTTGCCCTTTACGGGACGCCAGTGGACGTCCCCTTCCCCTTCC
GGGCAAGTAAACTTAGGGATTTTAATGCAATAAATAAATTTGTCCCCTT
ACGGGAATATAAATATTAGTGGCAGTTGCCTGCCAACTGCCGAGGCAAA
TAAATTTTAGTGGACAAATTTATTTATTGTTGCCTGCCAACTGCCGATA
TTTATATACTCCGAAGGAGGCAGTTGCCGGCCAACTGCCTGCCCCGCAG
TATTAACATAAGCAGTGTCGGTACCATTGCCACTGGCGTCCCGTTAGGT
GTTCGTAGCAGGCAGTGTCCTGCCACTGCCTCCTGTGGAGTATATAAAT
ATCCCTAAGTGTACTTGCCCGTAAAGGGAAAGGGAAGGGTATTTTATTT
ACTTTTTAGGTTTAGTAATAAATGAATCACAGGTAATTTCACTTTACAT
GCCATAAACTCTCTAATATACGTAAAGCTTCGCTCTGCGCAAGACTAAA
GTGCCCAAGCTCAAAGAACCTTTTCAAGAGTAAATAAATTCCCTTAAGG
GCTAATGAGCAAATTTAATGGTTATAGTTTTGTCAAGAAACGTTAGCAC
TGTTAGTACCAGGTTTAAAACATTTTTAATTTTTAATAATGCCAACAAA
CAATGTCGATGTAAGAAATGGATTTATTTTTTTAATAAATTTAATTTTA
TGCGTGAATCAATTCGAAGCGAATATGAAGTGAGCGGTTCAAAAAGAAC
AACTAACTATGGCTTTAGATATGTGAAATATCTATATAACTAAATTGCA
CACCCTTTTGTAAATAGAACCTAAAAAGGAAAAAAATTGAATTTAATAA
ATGACCCCTCAGCCAAAAGGTATGATTGGCAGTGCCTTTACTCGATAAA
CCCTTTGGAGATTTAATTGCCCTTAAAAGAAAAGACTAAAAATGTTTGC
TACCCAGGCAGTAGCGGGATTTGAAATACTCCGTACAGCAGACGTCCCC
TTACGGGACGTCAGTGGATATTTATATACTCCGAAGGAGGCAGTTGCCT
GCCAACTGCCGATATTTATATACTGCGATAAACTTTAGTTGCCCGAAGG
GGTTTACATACTAGGCAGTGGCGGTACCACTGCCACTGGCGTCCTCCTT
CGGAGTATGTAAACTACTTGCCCGAAGGGGAAGGAGGAAGCAGGCAGTG
GCGGTACCACTGCCCCTTACGGGATCCAATTGACGGGATCCGAATTCCG
TGAGCTCACGCGATCGCGAGGTACCCATATGACTAGTCATCTAGATAAA
TTTGCTAGTTTACATTATTTTTTATTTCTAAATATATAATATATTTAAA
TGTATTTAAAATTTTTCAACAATTTTTAAATTATATTTCCGGACAGATT
ATTTTAGGATCGTCAAAAGAAGTTACATTTATTTATATAAATGGCCCAA
AGCTCCGCCATCGATATCGGCATCACCAGCGCACAACGGGAGGAAATCG
CTGCTGAACTCAGCCGCCTCCTCGCAGATACGTACGTGTTGTACGGCAA
AACCCACGGTTTCCACTGGAACGTGACCGGGCCGATGTTCAACACATTG
CACCTGATGTTCATGGACCAGTACACCGAGTTGTGGAACGCCCTGGATG
TGATCGCTGAACGCATACGCGCCCTTGGCGTTTTGGCGCCCCATGGAGG
TTCCACCCTGGCCGGTTTGGCCTCGATCCAAGAAGCAGAGCAACAGCCT
GCCGCGCTCGACATGGTGCGTGAGCTGGTAACCGGCCATGAGGCCGTTG
CCCGTACGGCACGGAGCATCTTTCCTCTGGCAGAGGCTGCCAACGACGA
ACCCACTGCTGACCTGCTCACCCAACGACTTCAGATCCACGAAAAAACA
GCCTGGATGCTGCGCAGCCTGCTGGAAAACTAATAACTCGAGCCCGGGA
TGTGTAATTAAAATAAATATTTGGACACCATTAAGTTGTTTTCTTCTTA
AAGAGCCAATTTATTTTAATTACACATACATTATATATGTCATATTAGC
TATTTAATCTATTTACTTTAAAATAAAGGACGCCGTTAAACGTGTACGC
GTGCATCGGCTGGTACCATGCATGCGGCCGCAGTAGACTTTATTAGAGG
CAGTGTTTATATACCTAAACGTCAAAAGTCATTTTTATAACTGGTCTCA
AAATACCTATAAACCCATTGTTCTTCTCTTTTAGCTCTAAGAACAATCA
ATTTATAAATATATTTATTATTATGCTATAATATAAATACTATATAAAT
ACATTTACCTTTTTATAAATACATTTACCTTTTTTTTAATTTGCATGAT
TTTAATGCTTATGCTATCTTTTTTATTTAGTCCATAAAACCTTTAAAGG
ACCTTTTCTTATGGGATATTTATATTTTCCTAACAAAGCAATCGGCGTC
ATAAACTTTAGTTGCTTACGACGCCTGTGGACGTCCCCCCCTTCCCCTT
ACGGGCAAGTAAACTTAGGGATTTTAATGCAATAAATAAATTTGTCCTC
TTCGGGCAAATGAATTTTAGTATTTAAATATGACAAGGGTGAACCATTA
CTTTTGTTAACAAGTGATCTTACCACTCACTATTTTTGTTGAATTTTAA
ACTTATTTAAAATTCTCGAGAAAGATTTTAAAAATAAACTTTTTTAATC
TTTTATTTATTTTTTCTTTTATGGCAATGCGTACTCCAGAAGAACTTAG
TAATCTTATTAAAGATTTAATTGAACAATACACTCCAGAAGTGAAAATG
TCCATGGCTCGTGAAGCGGTGATCGCCGAAGTATCGACTCAACTATCAG
AGGTAGTTGGCGTCATCGAGCGCCATCTCGAACCGACGTTGCTGGCCGT
ACATTTGTACGGCTCCGCAGTGGATGGCGGCCTGAAGCCACACAGTGAT
ATTGATTTGCTGGTTACGGTGACCGTAAGGCTTGATGAAACAACGCGGC
GAGCTTTGATCAACGACCTTTTGGAAACTTCGGCTTCCCCTGGAGAGAG
CGAGATTCTCCGCGCTGTAGAAGTCACCATTGTTGTGCACGACGACATC
ATTCCGTGGCGTTATCCAGCTAAGCGCGAACTGCAATTTGGAGAATGGC
AGCGCAATGACATTCTTGCAGGTATCTTCGAGCCAGCCACGATCGACAT
TGATCTGGCTATCTTGCTGACAAAAGCAAGAGAACATAGCGTTGCCTTG
GTAGGTCCAGCGGCGGAGGAACTCTTTGATCCGGTTCCTGAACAGGATC
TATTTGAGGCGCTAAATGAAACCTTAACGCTATGGAACTCGCCGCCCGA
CTGGGCTGGCGATGAGCGAAATGTAGTGCTTACGTTGTCCCGCATTTGG
TACAGCGCAGTAACCGGCAAAATCGCGCCGAAGGATGTCGCTGCCGACT
GGGCAATGGAGCGCCTGCCGGCCCAGTATCAGCCCGTCATACTTGAAGC
TAGACAGGCTTATCTTGGACAAGAAGAAGATCGCTTGGCCTCGCGCGCA
GATCAGTTGGAAGAATTTGTCCACTACGTGAAAGGCGAGATCACTAAGG
TAGTTGGCAAATAACAGCTTGTACTCAAGCTCGTAACGAAGGTCGTGAC
CTTGCTCGTGAAGGTGGCGACGTAATTCGTTCAGCTTGTAAATGGTCTC
CAGAACTTGCTGCTGCATGTGAAGTTTGGAAAGAAATTAAATTCGAATT
TGATACTATTGACAAACTTTAATTTTTATTTTTCATGATGTTTATGTGA
ATAGCATAAACATCGTTTTTATTTTTTATGGTGTTTAGGTTAAATACCT
AAACATCATTTTACATTTTTAAAATTAAGTTCTAAAGTTATCTTTTGTT
TAAATTTGCCTGTGCTTTATAAATTACGATGTGCCAGAAAAATAAAATC
TTAGCTTTTTATTATAGAATTTATCTTTATGTATTATATTTTATAAGTA
ATAAAAGAAATAGTAACATACTAAAGCGGATGTAACTCAATCGGTAGAG
TGCGATCGCTAGCACTAGTCTCGAGGTTTAAACATGTACAAGCTTGGAT
CAATTGATTGGCTGCTGTGCAGCCCCAAAGTAGAAGGGTACGCCAGTGG
ACGTCAGTGGCAGGCAACTGCCTCCTTCGGAGTATATAAATATTGGCAG
TTGGCAGGCAACTGCCACTGACGTCCACTTAAATTTATTTGCCCGAAGG
GGACGTCCACTGGCGTACCGTAAGGGGAAGGGGACACCCACTGGCGTCC
CGAAAGGATTAGCCAATGGGCGAGGCCACTGCTTCAGCTACTAAAGTTT
TAAAGTGGTCTGTTGGACCATCAAATTCATATGGCATGTAATACTCCTA
ACGCTAATGCCTGGCTTTAGGGTCAGTTTACTGACCCAAAAACCTGTTA
ATAGCTTGTTAAACCCTTCTTTCCCTTTTGGAGTATATCACTATCACAA
AGGGGCGTCAGATGGAGTTAGTAAACTTTTTAAAGTGAACAAGAATCAC
AGATGTAATAAGCTTATTAAGTAAAGCTTAGAAAAATTGTTTTAATAAC
TAACAAAACTTTAAAAGTTCAAAAATTTTAATGTTATGTATAATATTTT
AATTAAAAAAACCATAACTATGTAAACCTTCTCCAATTAAATTTACACC
TAAATAACAAAACCAAACAACTAAAAAACCAACAGCAGCAATAATTGCC
GGTTTTTCACCTTCCCAACC

Enhanced Contamination Control and Separation Efficiency through Precise Iron Dosage and Feeding Strategies

Strategic periodic iron dosing can be used to both improve contamination control and enhance both magnetic and gravimetric separation techniques. Under this method, a minimal threshold iron requirement is temporarily exceeded through periodic dosing of soluble iron in chelated form. The soluble iron concentration in solution is periodically elevated above the minimal threshold until it is assimilated into algal biomass. The moderate to high cell density of algae with over-expressed high affinity iron transporters out-competes wild-type strains and contaminants for the brief period that iron is available.

Over time, iron is assimilated into algal biomass and accumulated to increasing levels. Just prior to harvesting, iron is dosed to very high levels, and is assimilated into algal biomass. This induces the over-accumulation of ferritin complexes thus achieving the conditions for magnetic separation of the algal biomass. The process of harvesting the biomass removes the iron from solution (assimilated in algal biomass) and the process continues while maintaining low iron concentrations. This dosing strategy may also allow ferritin to accumulate in the genetically modified algae in sufficient amounts to permit separation using gravimetric techniques.

Enhanced Lipid Extraction Using Heat Produced from Rapid Magnetic Field Polarity Reversal on Modified Microalgae

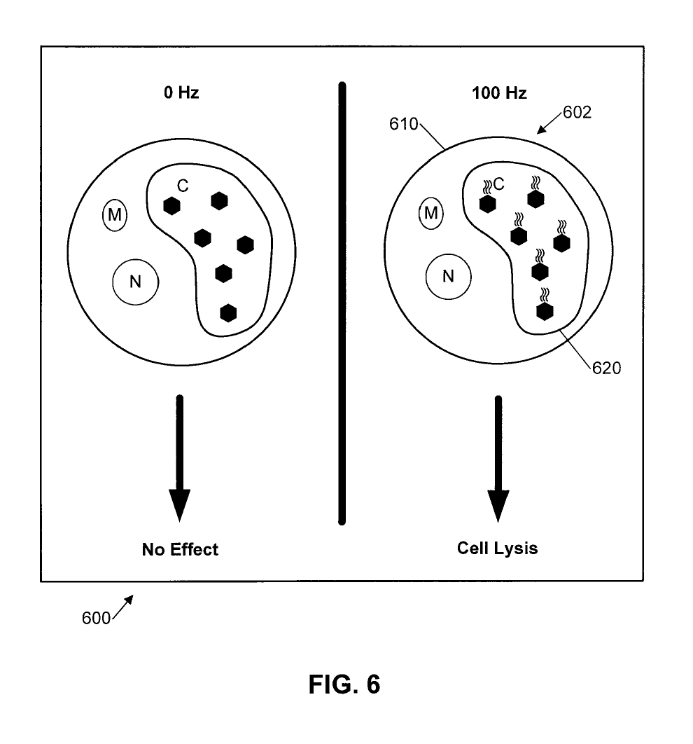

The energy required for lipid extraction for algae genetically modified to over-accumulate ferritin complexes can be reduced through the use of magnetic hysteresis. In the presence of a field of reversing magnetic polarity the dipole moment of paramagnetic particles switches orientations. At increased frequencies the internal resistance to this switching results in heat generation. At a frequency of or around 100 kHz, magnetic particles contained within or on the surface of algae could produce enough heat to cause cell deterioration and lysis. This is illustrated in FIG. 6. Cell lysis aids in the lipid extraction process as it liberates the oil from within the cell. Cell lysis through magnetic hysteresis would reduce the energy requirements for lipid extraction. This is achieved by focusing the energy expenditure on the target magnetically susceptible cells and not on the rest of the water and contaminating species.

Enhancement of Ma g netic Susce s tibilit and Contamination Control Throu h Iron Dosin Strategies

### Induced Magnetic Susceptibility During Growth in Excess Iron

Iron homeostasis is a significant constraint on the welfare of all forms of life. It is absolutely required in several protein complexes and is toxic when exposed to oxygen in the presence of a reduced metabolic state where many forms of reactive oxygen species can be produced. As a result, a mechanism for storing iron and controlling its availability is highly desirable. Surprisingly few mechanisms have been identified. Instead a nearly ubiquitous mechanism of storing iron in ferritin or bacterioferritin complexes is present. A variety of iron transporters and reductases act to contribute to the iron pool, but ferritin and bacterioferritin are largely responsible for regulating the availability of that iron pool.

One way to take advantage of the iron homeostasis mechanism for improved algal biofuels production is to grow the algae in excess iron. Iron concentrations at or in excess of 200 micromolar are inhibitory to photoautrophic growth of C. reinhardtii under full sunlight. However, it is not inhibitory to growth under low light or heterotrophic growth. Growth conditions where iron is spiked into the medium in the evening when the light levels are low permit optimal growth. The natural process of capturing iron and storing it enables photoautrophic growth when iron levels return to low levels. This condition enhances iron storage in the form of ferritin complexes. This increase in ferritin enhances magnetic susceptibility of the cell which enables magnetic manipulation.

With reference next to FIG. 2, an algal cell 200 is surrounded by chelated ferric/ferrous iron molecules 202 in solution 204. A ferric iron transporter 210 is illustrated. Ferritin complexes 220 and ferric reductases 222 are illustrated schematically. As shown, iron homeostasis under normal conditions is controlled by the ferritin, the iron transporters 210, and the iron reductases 222. The embodiments of the disclosure assist in promoting elevated iron homeostasis by enhancing the storage of iron in ferritin, and improving the iron transporters 210 and the iron reductases 222 mechanisms in manners to be described below.

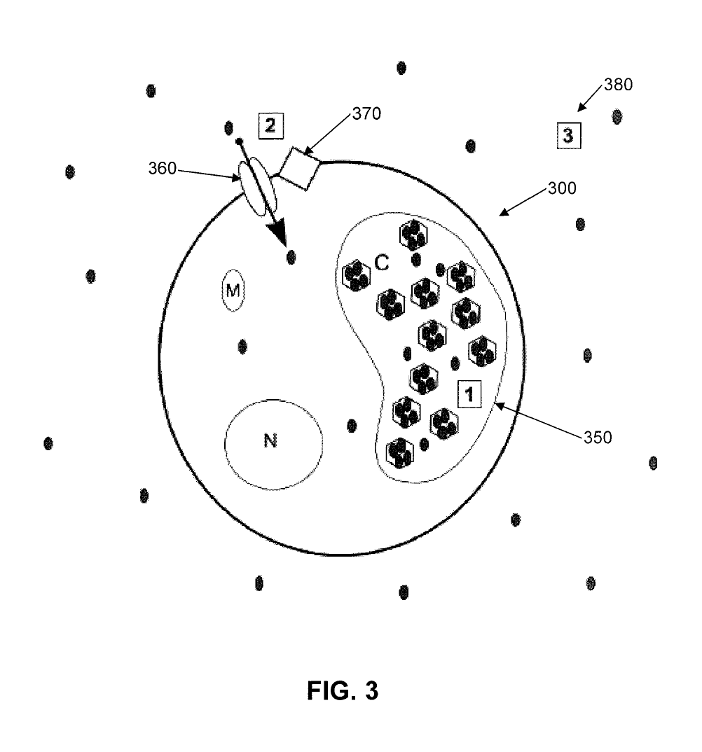

In particular, with reference next to FIG. 3, a schematic representation of iron homeostasis of an algal cell 300 in accordance with an example embodiment having enhanced magnetic susceptibility is shown. In accordance with the embodiments, high iron homeostasis is preferably achieved through at least three (3) routes, namely an over-expression of the ferritin gene 350 and resulting complexes, an over-expression of the iron transporter 360 and required iron reductase 370, and an increased availability of iron 380 in the surrounding media either continuously or in sporadic dosing.

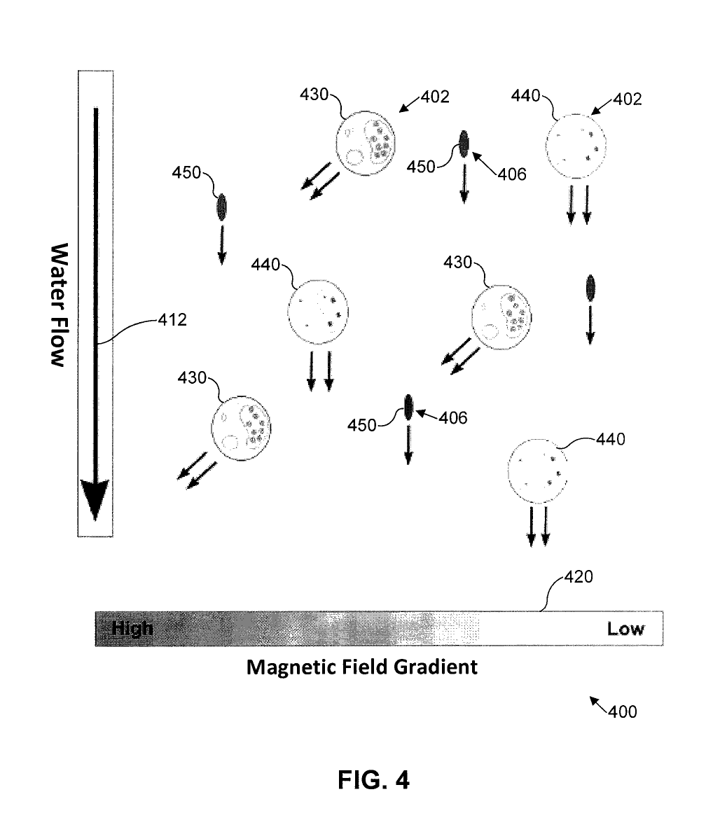

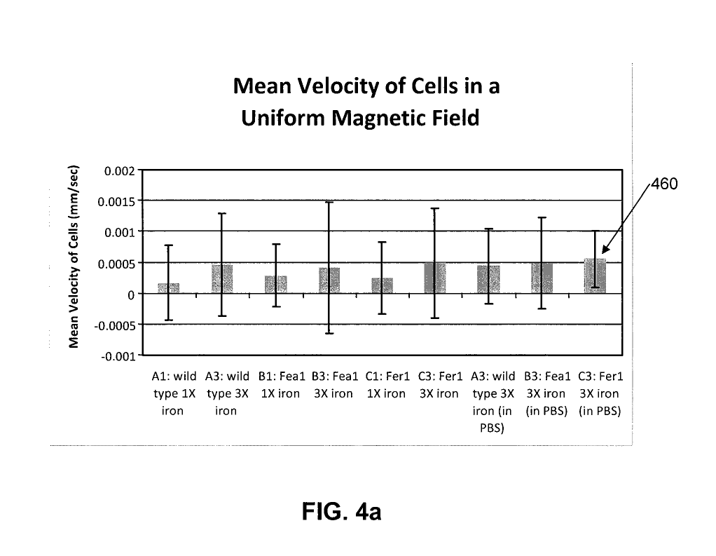

FIG. 4 presents a schematic illustration of a process 400 for separation of algae 402 with enhanced magnetic susceptibility from liquid media 404 and other organisms 406. As shown, the algae with enhanced magnetic susceptibility 402 are disposed in a flow 410 of a liquid such as, for example, a water flow. A magnetic field 420 is generated in a direction substantially transverse to a direction 412 of the water flow. Under these conditions, high ferritin algae 430 formed in accordance with any of the embodiments herein are reactive to the magnetic field and migrate in a direction transverse to the flow. On the other hand, under these conditions, low ferritin algae 440 not being formed in accordance with any of the embodiments herein, together with any other contaminants 450 are non-reactive to the magnetic field and therefore follow along without significant deviation with the fluid flow 410. Accordingly, by this process and system, high ferritin algae 430 formed in accordance with any of the embodiments herein are easily and efficiently separated from low ferritin algae 440 not being formed in accordance with any of the embodiments herein, together with any other contaminants 450.

FIG. 4a is a chart illustrating mean velocities of algae cells in a uniform magnetic field such as the field of FIG. 4. With reference in particular to FIG. 4a, algae strains in accordance with the embodiments are placed in a fluid bath and the mean velocity of the modified algae cells when exposed to a uniform magnetic field is measured. In accordance with another embodiment, an associated Cell Tracking Velocimetry (CTV) instrument is preferably used to help characterize the magnetic moment of selected one or more particle types in the solution. (Chalmers, et. al. Quantification of Non-Specific Binding of Magnetic Micro- and Nanoparticles Using Cell Tracking Velocimetry: Implication for Magnetic Cell Separation and Detection, Biotechnology and Bioengineering, Vol. 105, No. 6, Apr. 15, 2010). As illustrated in the chart, it is demonstrated in accordance with data collected that all of the cells move in the direction of the magnetic field. However only the Fer1 strain in accordance with the example embodiment herein moved fast enough to overcome their inherent random motility and gravitational settling. In the plots of FIG. 4a, it is to be appreciated that the error bars are greater than the mean velocity, except for the Fer1 strain 460 grown in elevated iron. While any of these strains could be separated using a stronger magnet than what is in this particular system, the most magnetically susceptible strain and condition was Fer1 grown in 3× the iron content of normal MHS.

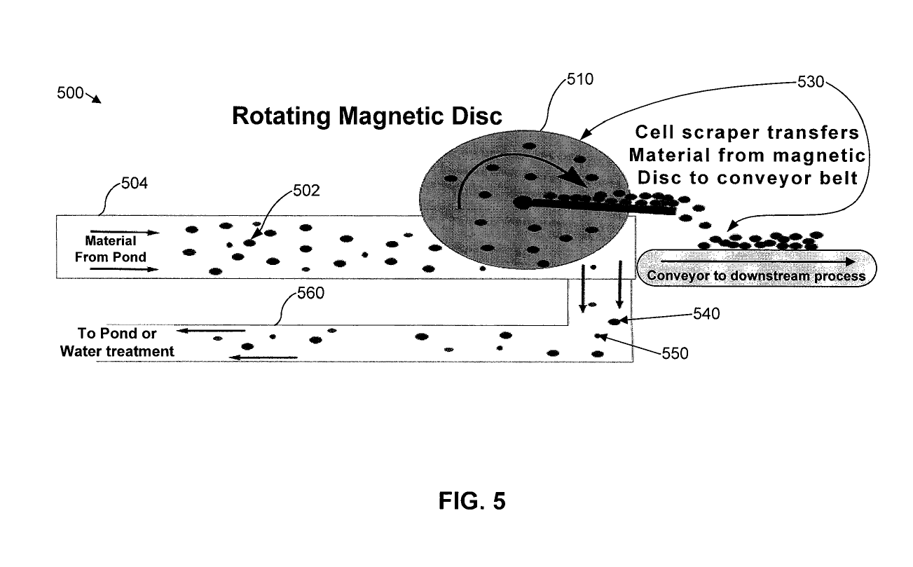

FIG. 5 shows a schematic illustration of a magnetic separator 500 in accordance with a further embodiment. As shown there, material 502 from a growth pond (not shown) is introduced to a rotating magnetic wheel 510 via a fluid chute 504. The high ferritin algae 530 are attracted to the wheel 510 because of the mutual magnetic properties of the wheel and algae. As discussed in connection with the embodiment of FIG. 4, high ferritin algae 530 formed in accordance with any of the embodiments herein are easily and efficiently separated from low ferritin algae 540 not being formed in accordance with any of the embodiments herein, together with any other contaminants 550 wherein the contaminants and low ferritin algae 540 pass by the wheel and continue on to a discharge chute 560 to be carried to a treatment plant or otherwise returned to the pond. In addition to the separation technology described above it is also potentially feasible in accordance with the embodiments to use other forms, systems or technologies of magnetic separation including for example superconducting electromagnetic filter separation or high intensity rare earth magnetic drum systems which can handle the high volumes and small particle sizes required for algal biofuels production.

FIG. 6 is a schematic comparative illustration 600 showing magnetically induced cell lysis 602 of algae 610 genetically modified in accordance with the embodiments herein to over-accumulate ferritin complexes 620 in accordance with the example embodiments.

Iron dosing strategy for control of contaminants

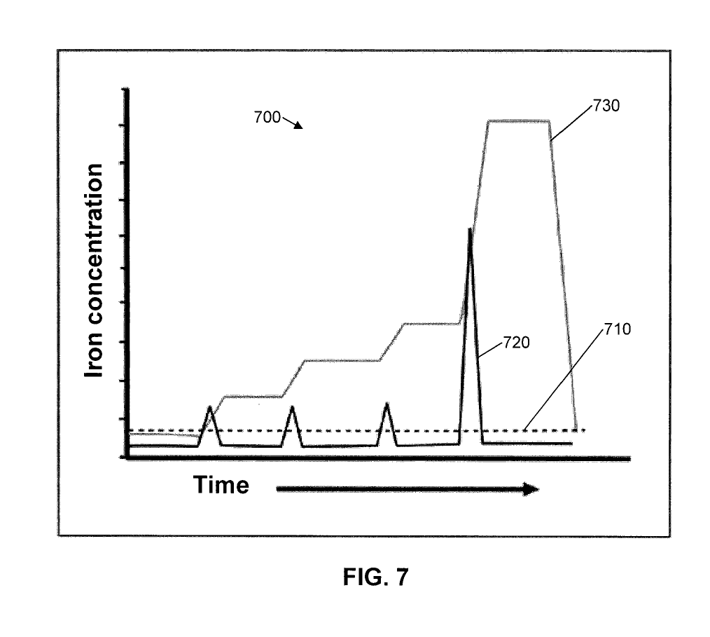

FIG. 7 illustrates a graph 700 showing enhancement of contamination control and separation efficiency through precise iron dosage and feeding strategies. A minimal threshold iron requirement 710 (dashed line) is temporally exceeded through periodic dosing of soluble iron in chelated form. The soluble iron concentration in solution 720 (black line) is periodically elevated above the minimal threshold until it is assimilated into biomass. The moderate to high cell density of algae with over-expressed high affinity iron transporters outcompetes wild type strains and contaminants for the brief period that iron is available. Iron is assimilated into algal biomass 720 (gray line) and accumulated to increasing levels. Just prior to harvesting, iron is dosed to very high levels, and is assimilated into algal biomass inducing the over-accumulation of ferritin complexes thus achieving the conditions for magnetic separation of the algal biomass. The process of harvesting the biomass removes the iron from solution (assimilated in algal biomass) and the process continues while maintaining low iron concentrations. This dosing strategy may also allow ferritin to accumulate in the genetically modified algae in sufficient amounts to permit separation using gravimetric techniques.

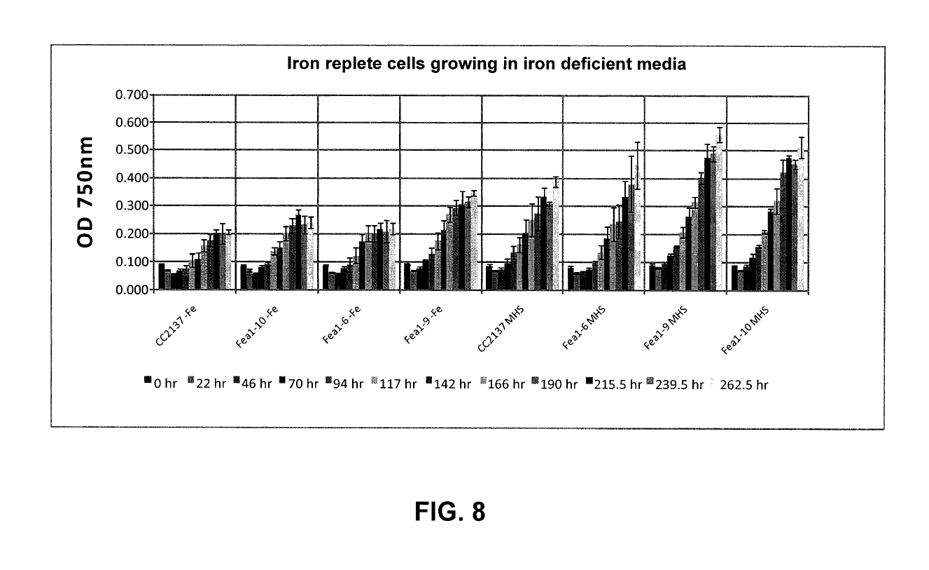

With reference next to FIG. 8, it is to be appreciated that the availability of iron in the growth media is a common constraint for both algae and contaminants in an open pond algal production scenario. The development of algal strains with enhanced iron uptake and storage makes it possible to inhibit growth of contaminants by limiting the bioavailability of iron. Algal strains with improved iron uptake or storage can be produced through the over-expression of ferritin, or iron transporters, and their corresponding reductase components. This is demonstrated in FIG. 8 where, as shown, several fea1 over-expression isolates were screened for growth under low iron conditions. In particular, it is to be noted that over-expression of the fea1 gene permits enhanced growth under low iron and low light conditions and can approximate growth of the wild-type under normal iron conditions with low illumination.

In this example, the high affinity iron storing algae are grown in medium with iron concentrations below the threshold required to maintain growth of the contaminating species. Periodically, additional iron is added to the medium to supply enough iron for algal growth. In a relatively short amount of time, however, this iron is biologically removed from the medium and assimilated into biomass as iron storage particles (ferritin complexes). Due in part to the high abundance of algae in the medium and largely to the high activity iron transport and storage of the engineered strain, it is able to out-compete contaminating strains and bacteria. The result is a scenario diagrammed in FIG. 7 where genetically modified algae are able to scavenge the trace iron and the temporarily excessively available iron that enables their growth while limiting the growth of competitors.

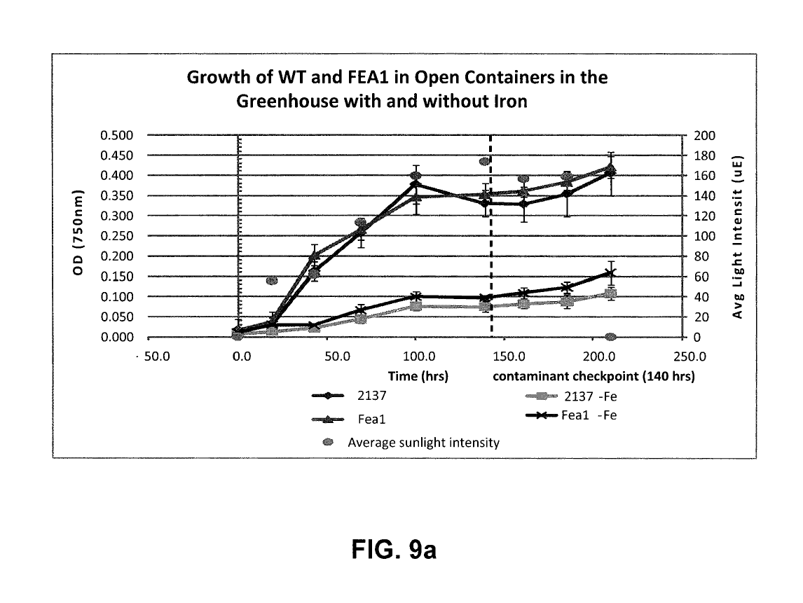

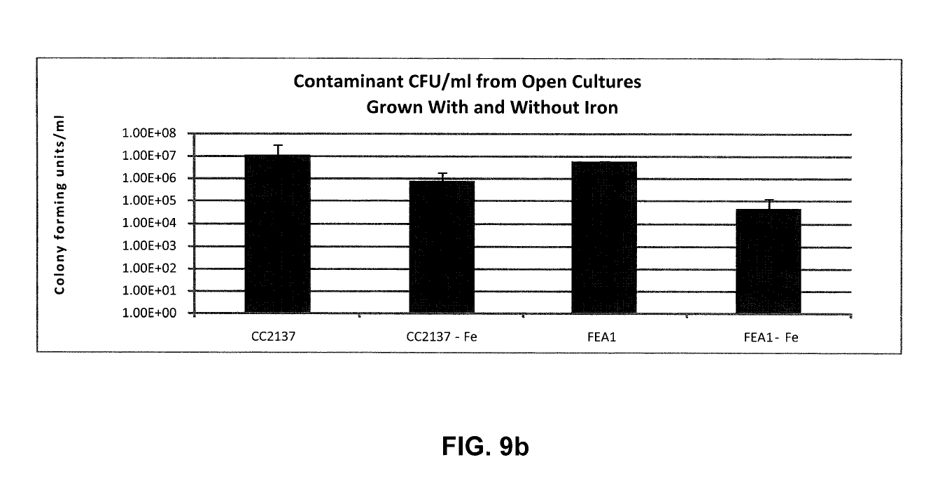

As demonstrated in FIGS. 9a and 9b, the modified strain over-expressing the fea1 gene under control of the psaD promoter and terminator shows improved growth under iron limiting conditions with reduced bacterial contamination when in open containers grown in a greenhouse environment (full sunlight). In this case, both strains were grown in open top vessels for a period of nine days. Samples were taken to measure optical density at 750 nm to demonstrate growth. After day six, samples were serially diluted and spread plated on tryptic soy agar to isolate single colonies. After one day's incubation, the colonies formed on the surface of the agar plates were counted (cfu/mL). Error bars represent the standard deviation across three replicate cultures of each strain at each condition.

In FIG. 9b, as shown, bacterial contamination is reduced by growing cultures under low iron conditions. Further improvements result when the algal strain is over-expressing the iron transporter gene fea1 whose gene product scavenges any iron introduced through airborne dust particle contamination or other methods.

Cell Lysis through Magnetic Hysteresis

With reference to FIG. 6, engineered strains of algae formed in accordance with any of the example embodiments herein are capable of producing excess ferritin complexes and storing iron in abundance inside these complexes making them paramagnetic. This can be achieved through any one or more of the examples provided above. Magnetically susceptible cells are then exposed to a magnetic field of reversing polarity of around 100 kHz. This field induces magnetic hyperthermia and cell damage. The result is damaged cell walls, which liberate the lipids contained within the cell. When used in combination with magnetic separation, this method reduces the energy requirements for cell lysis as energy is only applied to the target algae species, not contaminating species and excess water. Ferritin complexes of the novel algae of the embodiments are susceptible to magnetic hysteresis induced heat generation. Under conditions where ferritin complexes are present at high levels, it is possible to induce cell lysis from the heat generated from their resistance to rapid magnetic dipole switching. Cell lysis aids the lipid extraction process in the example embodiment by liberating the oil within the algal cells.

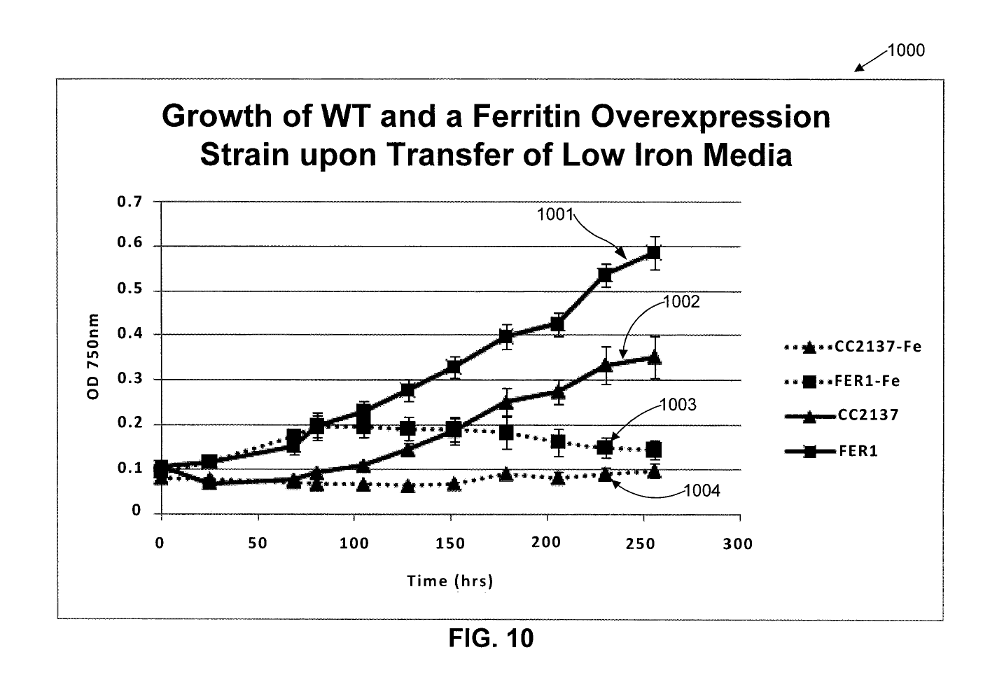

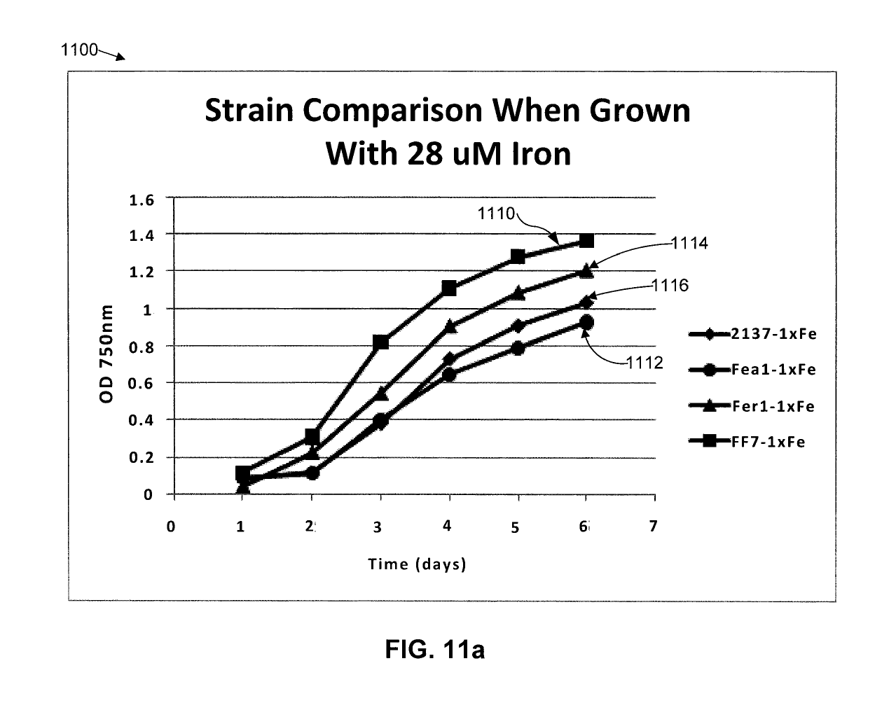

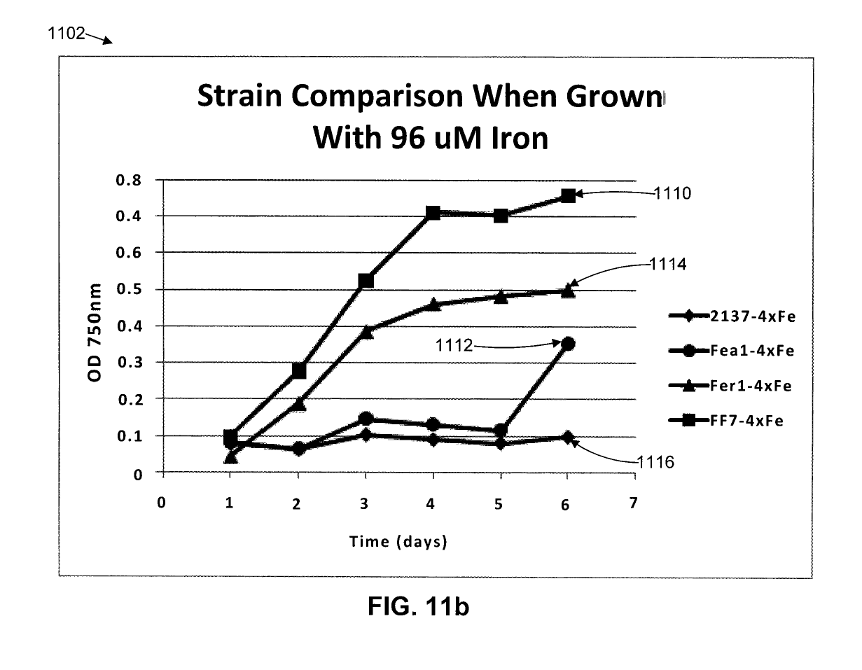

FIGS. 10, 11a, and 11b are charts with examples of one measure of increased productivity of modified strains grown under conditions of depleted iron and under excess iron. In both cases growth enhancement is demonstrated by increases in optical density which is often accepted as increases in biomass on a per volume basis. In FIG. 10 the fer1 overexpression strain 1003 tolerates the switch to low iron media and grows for several generations before inernal iron stores are depleted and growth becomes limited while the wild type strains growth 1004 is limited immediately upon transfer to low iron media. Note that the greater lag demonstrated by the wild-type strain 1002 is likely a result of the EDTA washes used to remove iron from the external surface of the cells. The actual growth maximum approximates that of the fer1 overexpression strain 1001. The impact of an extended lag period in growth is uncertain, whereas the impact of maximal growth rate is a certain impact on productivity.

FIGS. 11a and 11b demonstrate the additive or even synergistic impact of overexpression of multiple genes involved in iron assimilation on growth in media containing elevated iron. FIG. 11a illustrates a strain comparison graph 1100 in media containing 28 micromolar iron and FIG. 11b illustrates a strain comparison graph 1102 in media containing 96 micromolar iron. The FF7 strain 1110 co-expresses both fea1 and fer1 and confers greater tolerance to growth at high iron concentrations than either of the fea1 1112 or fer1 1114 overexpression lines, both of which are arguably better than Wild-type 1116 at growth under elevated iron conditions.

What is claimed is:

1. A method of separating genetically modified algae from an associated suspending liquid medium, the method comprising:
modifying wild-type algae so that one or more cellular iron assimilation components are enhanced under control of a constitutive or regulated promoter to produce genetically modified algae in an associated suspending liquid medium; and,
exposing the genetically modified algae to an associated magnetic field to magnetically separate the genetically modified algae from an associated suspending liquid medium.

2. The method of claim 1 wherein the modifying comprises:
modifying the algae to tolerate growth under high iron conditions through introduction of genes involved in alleviating iron stress including reactive oxygen species production.

3. The method of claim 2 wherein the introduction of genes comprises:
an introduction of reactive oxygen species responsive genes belonging to the radical scavenging enzymes selected from the group consisting of superoxide dismutase, peroxidase, catalase, and glutathione peroxidase.

4. The method of claim 2 wherein the introduction of genes comprises:
an introduction of reactive oxygen species responsive genes belonging to ferritin-like DPR or DPS genes which protect DNA from oxidative damage and store additional iron.

5. The method of claim 1 wherein the modifying comprises:
modifying the algae so that one or more cellular iron assimilation components are enhanced under control of a constitutive or regulated promoter selected from the group consisting of the native chloroplast or mitochondrial encoded promoter and terminator sequences.

6. The method of claim 1 wherein the modifying comprises:
modifying the algae so that one or more cellular iron assimilation components are enhanced under control of a constitutive or regulated promoter and terminator selected from the group consisting of those for photosynthesis core proteins, 16S rRNA, and chlorophyll biogenesis.

7. The method of claim 1 wherein the modifying comprises:
modifying the algae so that one or more cellular iron assimilation components are enhanced under the control of a regulated promoter triggered by one or more physical or chemical inducers.

8. The method of claim 1 wherein the enhanced cellular iron assimilation component is ferritin or bacterioferritin.

9. The method of claim 8 wherein the ferritin is selected from the group consisting of FER1 and FER2.

10. The method of claim 1 wherein the enhanced cellular iron assimilation components are an iron transporter and iron reductase.

11. The method of claim 10 wherein the iron transporter is selected from the group consisting of FEA1, FEA2, IRT1, and IRT2 and the iron reductase is FRE1.

12. The method of claim 10 wherein the promoter is the constitutively active highly expressed actin promoter.

13. The method of claim 1 wherein:
the magnetically separating comprises magnetically separating the genetically modified algae from an associated suspending liquid medium by an associated instrument for magnetic separation selected from the group consisting of a rare earth magnetic disc separator, a superconducting electromagnetic filter separator, a wet high intensity magnetic drum separator, a magnetic flow sorting system, and a magnetic microfluidic device.

14. The method of separating algae modified according to any of the preceding claims from the associated suspending liquid medium and enhancing contamination control within the culture, comprising an accelerated technique involving an iron dosing regimen which permits algae to accumulate iron into biomass giving it a growth advantage over contaminants.

15. The method of claim 14 wherein:
the associated suspending liquid medium containing the genetically modified algae is first dosed above a minimal threshold iron concentration that permits the genetically modified algae to survive but that is consumed quickly as to prevent other contaminating species from growing;
the minimal threshold is periodically exceeded to permit algae to quickly accumulate iron into biomass and deplete the medium of iron to levels below the minimal threshold; and
just prior to harvesting chelated iron is dosed to very high levels making the algae magnetically susceptible thus accelerating magnetic separation.

16. The method of claim 14 wherein the liquid medium containing the genetically modified algae is first dosed above a minimal threshold iron concentration that permits the genetically modified algae to quickly deplete the medium and out compete other contaminating organisms; the minimal threshold is periodically exceeded to permit algae to quickly accumulate iron into biomass and deplete the medium again; and just prior to harvesting is dosed to very high levels making the algae heavy and/or magnetically susceptible thus accelerating settling or enabling magnetic separation.

17. A method of algal lipid extraction, comprising exposing algae, modified according to one or more of claims 1 through 12, to a magnetic field of reversing polarity, which induces magnetic hysteresis and causes the cell to lyse, thereby liberating the cell's contents.

18. A magnetically enhanced alga prepared according to one or more of claims 1 through 12.

19. The method of claim 6 wherein the promoter is the RuBisCo large subunit (rbcL) and the terminator is the ATP synthase β subunit (atpB).

20. The method of claim 9 wherein the Fer1 gene is situated between the rbcL promoter and the atpB terminator such that they are operably linked to form an mRNA and protein expressing unit.

21. The method of claim 20 wherein the ferritin mRNA and protein expressing unit is situated in a plasmid next to a selective marker gene with its own promoter and terminator

22. The method of claim 21 wherein the ferritin mRNA and protein expressing unit along with the selective marker gene including its promoter and terminator are situated between two ≦600 nt regions of sequence with high homology to the sequence of the target strain for the purpose of targeting integration into an organellar genome.

23. The method of claims 1 and 11 wherein the iron transporter fea1 sequence is situated between the actin promoter and terminator sequence such that the fea1 gene and the promoter region are functionally linked to form a mRNA and protein expressing unit.

24. The method of claim 23 wherein the fea1 expressing unit is situated in the plasmid sequence near a selective marker gene with its respective promoter and terminator sequences.

25. The method of claim 24 wherein the constructs are linearized and used to transform the target algal strain through glass bead transformation, viral infection, biolistic bombardment, direct exposure to cell biomass, or any other common practice for transforming algae.

26. An algal strain with improved paramagnetic or ferromagnetic particle uptake and storage.

27. The algal strain of claim 26 wherein:
the algal strain is growth tolerant under high iron conditions through the introduction of genes involved in iron assimilation comprising mechanisms of iron uptake, storage, and alleviating iron stress including reactive oxygen species production.

28. The algal strain of claim 27 wherein:
the algal strain is tolerant to reactive oxygen species through the introduction of radical scavenging enzymes selected from the group comprising of superoxide dismutase, peroxidase, catalase, and glutathione peroxidase.

29. The algal strain of claim 27 wherein:
the algal strain is tolerant to reactive oxygen species through the introduction of genes belonging to ferritin-like DPR or DPS genes which protect DNA from oxidative damage and store additional iron.

30. The algal strain of claim 27 wherein:
the algal strain is configured to store iron through the introduction of one or more cellular iron assimilation components under the control of a constitutive or regulated promoter selected from the group consisting of the native chloroplast or mitochondrial encoded promoter and terminator sequences.

31. The algal strain of claim 27 wherein:
the algal strain is configured to store iron through the introduction of one or more cellular iron assimilation components under the control of a constitutive or regulated promoter and terminator selected from the group consisting of those for photosynthesis core proteins, 16S rRNA, and chlorophyll biogenesis.

32. The algal strain of claim 27 wherein:
the algal strain is configured to store iron through the introduction of one or more cellular iron assimilation components under the control of a regulated promoter triggered by light, specific carbohydrates, salt shock, or heat stress.

33. The algal strain of claim 27 wherein the enhanced cellular iron assimilation component is ferritin or bacterioferritin.

34. The algal strain of claim 33 wherein the ferritin is selected from the group consisting of FER1 and FER2.

35. The algal strain of claim 26 wherein the enhanced cellular iron assimilation components are an iron transporter and iron reductase.

36. The algal strain of claim 35 wherein the iron transporter is selected from the group consisting of FEA1, FEA2, IRT1, and IRT2 and the iron reductase is FRE1.

37. The algal strain of claim 36 wherein the promoter is the constitutively active highly expressed actin promoter.

38. The algal strain of claim 30 wherein the promoter is the RuBisCo large subunit (rbcL) and the terminator is the ATP synthase β subunit (atpB).

39. The algal strain of claims 34 and 38 wherein the Fer1 gene is situated between the rbcL promoter and the atpB terminator such that they are operably linked to form an mRNA and protein expressing unit.

40. The algal strain of claim 39 wherein the ferritin mRNA and protein expressing unit is situated in a plasmid next to a selective marker gene with its own promoter and terminator.

41. A transformation vector wherein the ferritin mRNA and protein expressing unit along with the selective marker gene including its promoter and terminator are situated between two ≧600 nt regions of sequence with high homology to the sequence of the target strain for the purpose of targeting integration into an organellar genome.

42. The algal strain of claims 26, 36, and 37 wherein the iron transporter fea1 sequence is situated between the actin promoter and terminator sequence such that the fea1 gene and the promoter region are functionally linked to form a mRNA and protein expressing unit.

43. The algal strain of claim 42 wherein the fea1 expressing unit is situated in the plasmid sequence near a selective marker gene with its respective promoter and terminator sequences.

44. The algal strain of claim 27 wherein the growth tolerance to high iron is through the introduction of multiple genes involved in iron assimilation.

45. A method of enhancing the productivity through the utilization of modified algal strains with enhanced paramagnetic and ferromagnetic particle assimilation.

46. The method of claim 45 wherein the modifications are comprised of that described in claims 2-12 and 19 through 24.

47. The method of claim 1 wherein the inducer is selected from the group consisting of light, specific carbohydrates, salt shock, and heat stress


SEQUENCE LISTING (3 sequences)

SEQ ID NO: 1 - DNA, 4883 bp, Chlamydomonas reinhardtii
cccttttggcaggacgtccctttccccttacgggatatttatatactcccatgttttgccctttacgggacgccagtggacgtccccttccccttccgggcaagtaaacttagggattttaatgcaataaataaatttgtccccttacgggaatataaatattagtggcagttgcctgccaactgccgaggcaaataaattttagtggacaaatttatttattgttgcctgccaactgccgatatttatatactccgaaggaggcagttgccggccaactgcctgccccgcagtattaacataagcagtgtcggtaccattgccactggcgtcccgttaggtgttcgtagcaggcagtgtcctgccactgcctcctgtggagtatataaatatccctaagtgtacttgcccgtaaagggaaagggaagggtattttatttactttttaggtttagtaataaatgaatcacaggtaatttcactttacatgccataaactctctaatatacgtaaagcttcgctctgcgcaagactaaagtgcccaagctcaaagaaccttttcaagagtaaataaattcccttaagggctaatgagcaaatttaatggttatagttttgtcaagaaacgttagcactgttagtaccaggtttaaaacatttttaatttttaataatgccaacaaacaatgtcgatgtaagaaatggatttatttttttaataaatttaattttatgcgtgaatcaattcgaagcgaatatgaagtgagcggttcaaaaagaacaactaactatggctttagatatgtgaaatatctatataactaaattgcacacccttttgtaaatagaacctaaaaaggaaaaaaattgaatttaataaatgacccctcagccaaaaggtatgattggcagtgcctttactcgataaaccctttggagatttaattgcccttaaaagaaaagactaaaaatgtttgctacccaggcagtagcgggatttgaaatactccgtacagcagacgtccccttacgggacgtcagtggatatttatatactccgaaggaggcagttgcctgccaactgccgatatttatatactgcgataaactttagttgcccgaaggggtttacatactaggcagtggcggtaccactgccactggcgtcctccttcggagtatgtaaactacttgcccgaaggggaaggaggaagcaggcagtggcggtaccactgccccttacgggatccaattgacgggatccgaattccgtgagctcacgcgatcgcgaggtacccatatgactagtcatctagataaatttgctagtttacattattttttatttctaaatatataatatatttaaatgtatttaaaatttttcaacaatttttaaattatatttccggacagattattttaggatcgtcaaaagaagttacatttatttatataaatggacaaaatcactggcattgttgttcagcctgctgttcagttctctgaagttcagtctgaacttgctactgttgataaaactaaccagaacatccagtcacttgcacgtgttgacttccacccagcttgtgaagcagctatcaacgaacaggtaaacatcgaatacaacgtttcttacctataccacgctctttgggcatacttcgaccgtgacaacgtagctctacctggccttgctgctttcttcaaagctggctctgaagaagaacgtgaacacgcagaacttcttatggaataccagaaccgtcgtggcggccgtgttgttctaggcgcaatctctatgcctgacctagacctttctgcttctgaaaaaggcgacgctctttacgcaatggaacttgctcttagcctagaaaaacttaacttccagaaacttcgtcagcttcactctgttgcagacgaacacggcgatgcttctatggctgacttcgtagaaggcgaattattaaacgaacaggttgaagctgttaaaaaagtttcagaatacgtatctcagcttcgtcgtgttggccagggcctaggcgtttaccagtttgacaaacagcttgcagctgaagtagcagctggtgctgcagcttaataactcgagcccgggatgtgtaattaaaataaatatttggacaccattaagttgttttcttcttaaagagccaatttattttaattacacatacattatatatgtcatattagctatttaatctatttactttaaaataaaggacgccgttaaacgtgtacgcgtgcatcggctggtaccatgcatgcggccgcagtagactttattagaggcagtgtttatatacctaaacgtcaaaagtcatttttataactggtctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttatggcaatgcgtactccagaagaacttagtaatcttattaaagatttaattgaacaatacactccagaagtgaaaatgtccatggctcgtgaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataacagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttattttttatggtgtttaggttaaatacctaaacatcattttacatttttaaaattaagttctaaagttatcttttgtttaaatttgcctgtgctttataaattacgatgtgccagaaaaataaaatcttagctttttattatagaatttatctttatgtattatattttataagtaataaaagaaatagtaacatactaaagcggatgtaactcaatcggtagagtgcgatcgctagcactagtctcgaggtttaaacatgtacaagcttggatcaattgattggctgctgtgcagccccaaagtagaagggtacgccagtggacgtcagtggcaggcaactgcctccttcggagtatataaatattggcagttggcaggcaactgccactgacgtccacttaaatttatttgcccgaaggggacgtccactggcgtaccgtaaggggaaggggacacccactggcgtcccgaaaggattagccaatgggcgaggccactgcttcagctactaaagttttaaagtggtctgttggaccatcaaattcatatggcatgtaatactcctaacgctaatgcctggctttagggtcagtttactgacccaaaaacctgttaatagcttgttaaacccttctttcccttttggagtatatcactatcacaaaggggcgtcagatggagttagtaaactttttaaagtgaacaagaatcacagatgtaataagcttattaagtaaagcttagaaaaattgttttaataactaacaaaactttaaaagttcaaaaattttaatgttatgtataatattttaattaaaaaaaccataactatgtaaaccttctccaattaaatttacacctaaataacaaaaccaaacaactaaaaaaccaacagcagcaataattgccggtttttcaccttcccaacca

SEQ ID NO: 2 - DNA, 4331 bp, Chlamydomonas reinhardtii
  misc_feature (2146)..(2146): n is a, c, g, or t
ctgggtacccgcttcaaatacgcccagcccgcccatggagaaagaggccaaaatcaacggaggatcgttacaaccaacaaaattgcaaaactcctccgctttttacgtgttgaaaaagactgatcagcacgaaacggggagctaagctaccgcttcagcacttgagagcagtatcttccatccaccgccgttcgtcagggggcaaggctcagatcaacgagcgcctccatttacacggagcggggatcccaacgtccacactgtgctgtcacccacgcgacgcaaccctacccagccaccaacaccatcaggtccctcagaagaactcgtccaacagccggtaaaacgccagcttttcctccgataccgccccatcccacccgcgcccgtactcccgcaggaacgccgcggaacactccggcccgaaccacgggtcctcctcgtgggccagctcgcgcagcaccagcgcgagatcggagtgccggtccgcacggccgacccgccccacgtcgatcagcccggtcacctcgcaggtacgagggtcgagcagcacgttgtccgggcacaggtgaccgtggcaaaccgccagatcctcgtccgcaggccgagtccgctccagctcggcgagaagccgctcccccgaccaccccttccgctcctcgtccagatcctccaagtcgacgctcccttcagcgacagcacgggccgcctgcggcaccgtcaccgcgagactgcgatcgaacggacaccgctcccagtccagcgcgtgcagcgaacgagcgagccccgcgagcgccaccgccacgtccagccgctgctcccgcggccaccgcgcactggccggacgccccggaaccgcttcggtgaccaaccaggcgaccctctcgtccccaccaccctccacaacacgaggtacgggaatccccacctccgccaaccacaccagccgctcagcctcacccaacaagcccaccccggcccccagagctgccaccttgacaaacaactcccgcccaccaccccgaagccgataaacaccagcccccgaggccccatcctccacaacaacccactcacaaccgggataccgaccccgcagtgcacgcaacgcatcgtccatgcttcgaaattcttcagcaccggggagggcggagtggccatcctgcaaatggaaacggcgacgcagggttagatgctgcttgagacagcgacagaggagccaaaagccttcgtcgacacaatgcgggcgttgcaagtcaaatctgcaagcacgctgcctgatccgccgggcttgctcgtcgactcacctggccattttaagatgttgagtgacttctcttgtaaaaaagtaaagaacataggccccctggccggtttatcaggagggcaccgctccaggggctgcatgcgaactgcttgcattggcgcctagcctttgtgggccagggggcttccggataagggttgcaagtgctcaaataccccatcaaacatcatcctggtttggctgcgctccttctggcgcgcccggcatgcaagcttgatgggatcttaagctagctgagtggttatgtatagcggcagaatagtcgcgtatgtataagtgctcgtttgtcgctgaaagtggaggtcaccgttcggggtcgcgggcttttataccggatgggtgccgccagcgggccgtatggcgccttctggacgccgcgcgccccatcgcggcccttccagagcttcccgcgccctcatagcccgccaaatcagtcctgtagcttcatacaaacatacgcaccaatcatgtcaagcctcagcgagctccccgctcgagcacacacctgcccgtctgcctgacaggaagtgaacgcatgtcgagggaggcctcaccaatcgtcacacgagccctcgtcagaaacacgtctccgccacgctctccctctcacggccgaccccgcagcccttttgccctttcctaggccaccgacaggacccaggcgctctcagcatgcctcaacaacccgtactcgtgccagcggtgcccttgtgctggtgatcgcttggaagcgcatgcgaagacgaaggggcggagcaggcggcctggctgttcgaagggctcgccgccagttcgggtgcctttctccacgcgcgcctnccacacctaccgatgcgtgaaggcaggcaaatgctcatgtttgcccgaactcggagtccttaaaaagccgcttcttgtcgtcgttccgagacatgttagcagatcgcagtgccacctttcctgacgcgctcggccccatattcggacgcaattgtcatttgtagcacaattggagcaaatctggcgaggcagtaggcttttaagttgcaaggcgagagagcaaagtgggacgcggcgtgattattggtatttacgcgacggcccggcgcgttagcggcccttcccccaggccagggacgattatgtatcaatattgttgcgttcgggcactcgtgcgagggctcctgcgggctggggagggggatctgggaattggaggtacgaccgagatggcttgctcggggggaggtttcctcgccgagcaagccagggttaggtgttgcgctcttgactcgttgtgcattctaggaccccactgctactcacaacaagcccatatgtcggtcggatttctggtcctcgcgctgggcgcgcttgtcgtggcgacggcggactacaaggacgacgacgacaagcagcccacgacgactggcactcgcttcgagggtttctcttacgcgggcaatgtcattggctatgtgaacatgacgatggactactgcgacatcaaggccgccatggctgctggcaacttcaccgaggctctgtccatctactctactggcaagaactcgttctctggcctggcgcgccgcaccttcttccgcttcgcctcgtacatcaccgccaacggctccgtggagccgctgcacgactccatcctggccggcaaggacacgtcctccctggacgccgccatccgggctgccctggccgacggcaaggccaccctggccgccggtctgatccaggtgggcacgctcaagtaccacctgcacgaggtggatgaggcctacaacaagatcaagacctacctggctgacggcaccggcaacctcaccaacctggtttctgacgcctcgggcgcaccccacaacgtggatgaggcctgggcgctgtgggccggcggcgccgccaacaactgcggcaccctgtccggctgggcctcctccctgggcgccgccatgggcaccaccttcctcggcaagagctatgtcaacaccgccatgatcaacaccgtcaatgagatgctggccgccgcccgcctgtctaccctgaacatccaagcctacgacgccgcgcgcacgaacgaggtccgactgctgaccctgctgggcctgcagggcgtgtccgtggccgcgtacaccgctgacgccgccgccgcctgcaagcgccccgccgccgaggtggaggatgccaagaccatgatcgccgtgcactgggcgtacctggagcctatgctcaagctgcgcaacttcaaggcctccgccgtgaccgagctgcaccaccagctcaccgcctccaagctgagctacaagaaggtggccgccgcggtgaagggcgtgctgtcggctatgggccgccgctccagcgagctgggtgccccgcagtccgccatcattgccgccaactggaagtgcagctccaagaccctgcgcagcattgcgtaatctagatggcagcagctggaccgcctgtaccatggagaagagctttacttgccgggatggccgatttcgctgattgatacgggatcggagctcggaggctttcgcgctaggggctaggcgaagggcagtggtgaccagggtcggtgtggggtcggcccacggtcaattagccacaggaggatcagggggaggtaggcacgtcgacttggtttgcgaccccgcagttttggcggacgtgctgttgtagatgttagcgtgtgcgtgagccagtggccaacgtgccacacccattgagaagaccaaccaacttactggcaatatctgccaatgccatactgcatgtaatggccaggccatgtgagagtttgccgtgcctgcgcgcgccccgggggcgcagtttagctgaccagccgtgggatgatgcacgcatttgcaaggacagggtaatcacagcagcaacatggtgggcttaggacagctgtgggtcagtggacggacggcaggggagggacggcgcagctcgggagacagggggagacagcgtgactgtgcaatgcggccgccaccgcggtgga

SEQ ID NO: 3 - DNA, 4724 bp, Chlamydomonas reinhardtii
gcccttttggcaggacgtccctttccccttacgggatatttatatactcccatgttttgccctttacgggacgccagtggacgtccccttccccttccgggcaagtaaacttagggattttaatgcaataaataaatttgtccccttacgggaatataaatattagtggcagttgcctgccaactgccgaggcaaataaattttagtggacaaatttatttattgttgcctgccaactgccgatatttatatactccgaaggaggcagttgccggccaactgcctgccccgcagtattaacataagcagtgtcggtaccattgccactggcgtcccgttaggtgttcgtagcaggcagtgtcctgccactgcctcctgtggagtatataaatatccctaagtgtacttgcccgtaaagggaaagggaagggtattttatttactttttaggtttagtaataaatgaatcacaggtaatttcactttacatgccataaactctctaatatacgtaaagcttcgctctgcgcaagactaaagtgcccaagctcaaagaaccttttcaagagtaaataaattcccttaagggctaatgagcaaatttaatggttatagttttgtcaagaaacgttagcactgttagtaccaggtttaaaacatttttaatttttaataatgccaacaaacaatgtcgatgtaagaaatggatttatttttttaataaatttaattttatgcgtgaatcaattcgaagcgaatatgaagtgagcggttcaaaaagaacaactaactatggctttagatatgtgaaatatctatataactaaattgcacacccttttgtaaatagaacctaaaaaggaaaaaaattgaatttaataaatgacccctcagccaaaaggtatgattggcagtgcctttactcgataaaccctttggagatttaattgcccttaaaagaaaagactaaaaatgtttgctacccaggcagtagcgggatttgaaatactccgtacagcagacgtccccttacgggacgtcagtggatatttatatactccgaaggaggcagttgcctgccaactgccgatatttatatactgcgataaactttagttgcccgaaggggtttacatactaggcagtggcggtaccactgccactggcgtcctccttcggagtatgtaaactacttgcccgaaggggaaggaggaagcaggcagtggcggtaccactgccccttacgggatccaattgacgggatccgaattccgtgagctcacgcgatcgcgaggtacccatatgactagtcatctagataaatttgctagtttacattattttttatttctaaatatataatatatttaaatgtatttaaaatttttcaacaatttttaaattatatttccggacagattattttaggatcgtcaaaagaagttacatttatttatataaatggcccaaagctccgccatcgatatcggcatcaccagcgcacaacgggaggaaatcgctgctgaactcagccgcctcctcgcagatacgtacgtgttgtacggcaaaacccacggtttccactggaacgtgaccgggccgatgttcaacacattgcacctgatgttcatggaccagtacaccgagttgtggaacgccctggatgtgatcgctgaacgcatacgcgcccttggcgttttggcgccccatggaggttccaccctggccggtttggcctcgatccaagaagcagagcaacagcctgccgcgctcgacatggtgcgtgagctggtaaccggccatgaggccgttgcccgtacggcacggagcatctttcctctggcagaggctgccaacgacgaacccactgctgacctgctcacccaacgacttcagatccacgaaaaaacagcctggatgctgcgcagcctgctggaaaactaataactcgagcccgggatgtgtaattaaaataaatatttggacaccattaagttgttttcttcttaaagagccaatttattttaattacacatacattatatatgtcatattagctatttaatctatttactttaaaataaaggacgccgttaaacgtgtacgcgtgcatcggctggtaccatgcatgcggccgcagtagactttattagaggcagtgtttatatacctaaacgtcaaaagtcatttttataactggtctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttatggcaatgcgtactccagaagaacttagtaatcttattaaagatttaattgaacaatacactccagaagtgaaaatgtccatggctcgtgaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataacagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttattttttatggtgtttaggttaaatacctaaacatcattttacatttttaaaattaagttctaaagttatcttttgtttaaatttgcctgtgctttataaattacgatgtgccagaaaaataaaatcttagctttttattatagaatttatctttatgtattatattttataagtaataaaagaaatagtaacatactaaagcggatgtaactcaatcggtagagtgcgatcgctagcactagtctcgaggtttaaacatgtacaagcttggatcaattgattggctgctgtgcagccccaaagtagaagggtacgccagtggacgtcagtggcaggcaactgcctccttcggagtatataaatattggcagttggcaggcaactgccactgacgtccacttaaatttatttgcccgaaggggacgtccactggcgtaccgtaaggggaaggggacacccactggcgtcccgaaaggattagccaatgggcgaggccactgcttcagctactaaagttttaaagtggtctgttggaccatcaaattcatatggcatgtaatactcctaacgctaatgcctggctttagggtcagtttactgacccaaaaacctgttaatagcttgttaaacccttctttcccttttggagtatatcactatcacaaaggggcgtcagatggagttagtaaactttttaaagtgaacaagaatcacagatgtaataagcttattaagtaaagcttagaaaaattgttttaataactaacaaaactttaaaagttcaaaaattttaatgttatgtataatattttaattaaaaaaaccataactatgtaaaccttctccaattaaatttacacctaaataacaaaaccaaacaactaaaaaaccaacagcagcaataattgccggtttttcaccttcccaacc